(19) United States
(12) Patent Application Publication
(10) Pub. No.: US 2024/0393934 A1
(43) Pub. Date: Nov. 28, 2024

(54) Biometric and user-interaction analysis and recommendation system

(71) Applicant: Medibio Limited, Savage, MN (US)

(72) Inventors: Massimiliano Grassi, Milan (IT); Silvia Daccò, Cabiate (IT); Archie Defillo, Savage, MN (US); Sarah Mellen, Detroit, MI (US); Mark Anderson, Plymouth, MN (US)

(21) Appl. No.: 18/691,239

(22) PCT Filed: Oct. 3, 2022
(86) PCT No.: PCT/US2022/045540

§ 371 Date: Mar. 12, 2024

Related U.S. Application Data
(60) Provisional application No. 63/251,513, filed on Oct. 1, 2021.

(51) Int. Cl. G06F 3/04847; G06F 3/04883; G06F 9/451

(52) U.S. Cl. CPC G06F 3/04847; G06F 3/04883; G06F 9/451

(57) ABSTRACT

This document generally relates to a system that receives biometric and user-interaction data, analyzes such data, and provides responsive recommendations.

### CROSS-REFERENCE TO RELATED APPLICATIONS

This application claims the benefit of U.S. Provisional Application Ser. No. 63/251,513, filed Oct. 1, 2021.

### TECHNICAL FIELD

This document generally relates to a system that receives biometric and user-interaction data, analyzes such data, and provides responsive recommendations.

### BACKGROUND

Some user-wearable devices monitor user biometrics and present informative data to users. For example, a user may wear a computerized watch that includes sensors which measure device movement and orientation, and that measure a pulse rate of a heart of the user.

### SUMMARY

This document describes techniques, methods, systems, and other mechanisms for receiving biometric and user-interaction data, analyzing such data, and providing responsive recommendations.

The details of one or more implementations are set forth in the accompanying drawings and the description below. Other features, objects, and advantages will be apparent from the description and drawings, and from the claims.

### DESCRIPTION OF DRAWINGS

FIG. 1 shows an example computing system in which the technology described in this application may be implemented.

FIGS. 2A-B and 3 show initial user interfaces of an application program.

FIGS. 4A-F show interface screen presented after a user indicates that the user is going to sleep.

FIGS. 5A-F show interface screens for an assessment, and interface screens for results to the assessment.

FIGS. 6A-C show interface screens for a biometrics module.

FIGS. 7A-C show interface screens for an Odyssey interactive experience.

FIGS. 8A-D show interface screens for a personality sub-module.

FIGS. 9A-E show interface screens for results to the personality sub-module.

FIGS. 10A-L show interface screens for an emotional intelligence sub-module.

FIGS. 11A-J show interface screens for results to the emotional intelligence sub-module.

FIGS. 12A-E show interface screens for an awareness module.

FIGS. 13A-E show interface screens for a distress/eustress interface.

FIGS. 14A-M show interface screens for an occupational burnout workflow.

FIGS. 15A-C show interface screens for results to the occupational burnout workflow.

FIGS. 16A-O show interface screens for a misalignment module.

FIGS. 17A-D show interface screens for results to the misalignment module.

FIG. 18 shows example reminder notifications.

FIG. 19 is a conceptual diagram of a system that may be used to implement the systems and methods described in this document.

FIG. 20 is a block diagram of computing devices that may be used to implement the systems and methods described in this document, as either a client or as a server or plurality of servers.

Like reference symbols in the various drawings indicate like elements.

### DETAILED DESCRIPTION

This document generally describes a system and operations for receiving biometric and user-interaction data, analyzing such data, and providing responsive recommendations. For example, a computing device such as a mobile telephone may include an application program that monitors user biometrics and provides recommendations to the user regarding behavioral modification based on such user biometrics. The application program may also prompt the user to answer questions regarding personality characteristics, stressors in the user's life, and coping mechanisms for such stressors, and may provide behavioral recommendations to the user based on such information.

Stress is often considered one of the leading causes of human illness and disease. Indeed, the prevalence of stress in primary care is high: 60-80% of visits may have a stress-related component. JAMA Intern Med. 2013; 173 (1): 76-77. doi: 10.1001/2013. Understanding and identifying negative stress before it becomes a more serious condition can prevent both mental and physiological consequences.

The application program described herein provides an interactive user experience that helps a user understand the user's personal stress response and realign unhelpful thought patterns. The application program also presents interactive surveys with questions designed to measure the stress a user is experiencing, and reminds a user to regularly take re-take such surveys so that users are able to discern the trajectories of certain stressors and behaviors.

The application program described herein also assists users in identifying maladaptive behaviors, which are behaviors that a user engages in to cope with specific situations, but with dysfunctional results. Maladaptive behaviors stop a user from adapting to new or difficult circumstances, and can lead to emotional, social, and health problems. Such behaviors can start after a major life change, illness, or traumatic event. They can also be a habit a user picked up at an early age.

Examples of maladaptive behaviors include not making eye contact during a conversation, speaking too softly or not at all, spurs of anger, voluntary not seeking information or withholding support when needed, performing self-harm, and having passive-aggressive episodes. The application program described herein can help users identify maladaptive behaviors and replace them with more productive behaviors. Avoidance behavior and escape coping are maladaptive forms of coping in which a person changes their behavior to avoid thinking about, feeling, or doing difficult things. Avoidance coping involves trying to avoid stressors rather than dealing with them.

The following disclosure describes such an application program, including the unique manners in which users interact with computer while the application program is executing, and how the application program responds to signals indicative of user interactions and hardware biometric sensors.

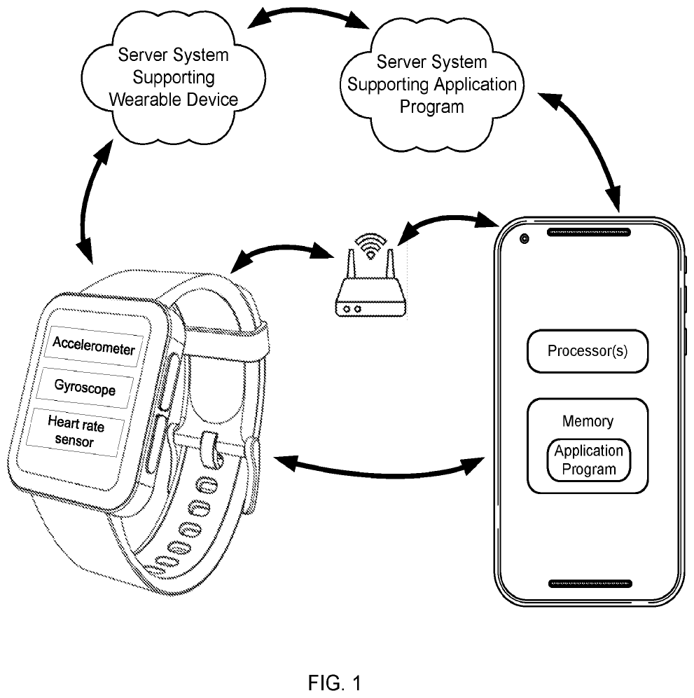

An example computing system in which the technology described in this application may be implemented is shown in FIGS. 1 and 19-20. The computing system includes a user-wearable computing device (e.g., a smartwatch) that includes various biometric sensors (e.g., one or more accelerometers, gyroscopes, and heart rate sensors).

The user-wearable computing device may wirelessly communicate with a computing device on which the application program is installed multiple manners: (1) directly (e.g., via Bluetooth), (2) via a shared router (e.g., with each device communicating with the router using Ethernet), (3) or via the internet (e.g., using cellular networks to interact with a server system that supports each device, and which may communicate with each other). Communications between a wearable device and a computing device in communication therewith may include unprocessed biometrics data or information derived therefrom (e.g., an indication that the user is in a “low sleep state” or is “running” based on analysis by the wearable device of the biometric data).

Analysis and determinations described herein may be performed at various different locations of the computing system, for example, by the application program at the user computing device or at the server system that supports the application program, and which may include databases and repositories that store various types of relevant data. The application program may execute on the computing device based on one or more processors executing instructions stored in computer-readable memory.

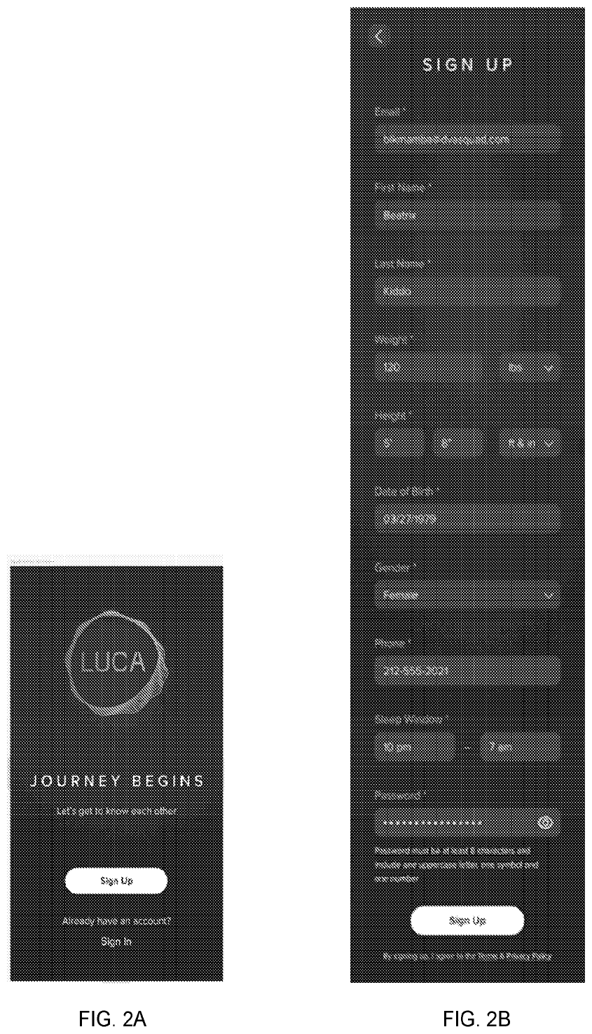

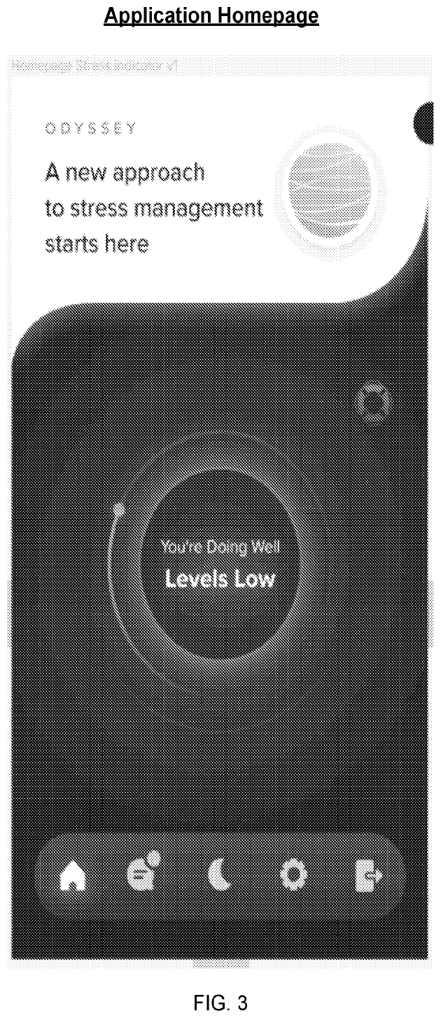

The application program may be downloaded to a user computing device, for example, from the APPLE APPSTORE or the GOOGLE PLAY STORE. After installation and launch, the application program may display the user interface screen shown in FIG. 2A.

Responsive to user input that selects the “Sign Up” interface element in the FIG. 2A interface, the computing device transitions its touchscreen display device from that shown in the FIG. 2A interface to the “Sign Up” user interface screen shown in FIG. 2B.

### Application Homepage

The computing device presents the interface screen shown in FIG. 3 after user sign up (or immediately after launch if the user has previously signed up for and is logged into a user account).

Selecting the curved pop-up banner at the top of the FIG. 3 user interface causes the user device to navigate to the “Odyssey” portion of the application program, which prompts an interactive user experience to assist the user in identifying their personality and emotional intelligence, stressors, and copying mechanisms for such stressors.

The circle interface element in the middle of the FIG. 3 interface display indicates, based on metrics described in more detail throughout this disclosure, which of the following categories the user's current stress level falls within:
“You're Doing Well—Levels Low”
“Take Notice—Levels Medium”
“Attention—Levels High”
   Selecting the circle interface element causes the user interface to navigate to a portion of the application program that displays metrics determined based on user biometric data recorded at least partially using computerized wearable devices (e.g., a smartwatch that can monitor user biometrics).

The five icons at the bottom of the FIG. 3 homepage interface are user selectable to perform the following actions:
Home icon: Navigate to the home page.
Chat bubble icon: Navigate to an Assessment that a user can regularly complete.
Moon icon: User selection designates to the application program that the user is now attempting to fall asleep.
Gear icon: Navigates to a settings page.
Exit door: Exits the application program.

### Sleep Cycle

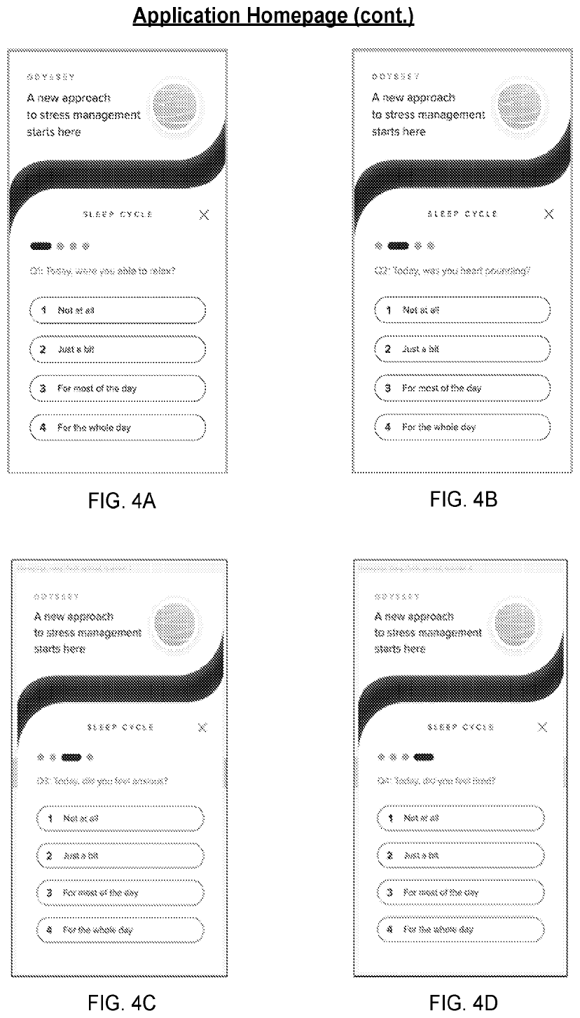

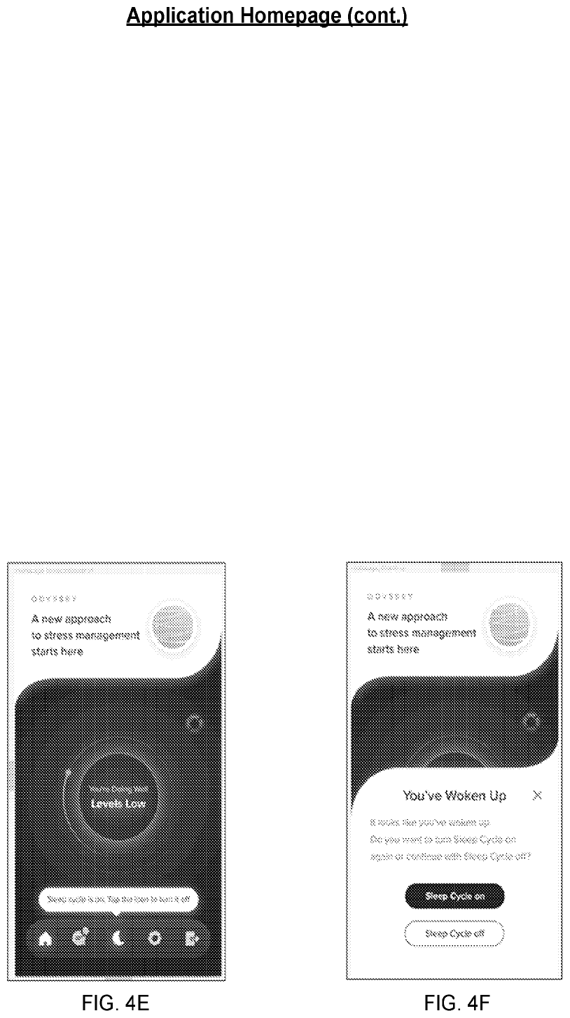

As described above, users are meant to select the moon icon when laying down to bed and intending to go to sleep (e.g., after done reading a book while laying down in bed). In response, the application program (1) records the current time as a time at which the user started trying to sleep, and (2) presents the series of interactive screens shown in FIGS. 4A-D, which present four questions and possible answers. Each numbered interface element is selectable by user contact with a portion of a touchscreen at which the interface element is displayed. User input may swipe left/right to switch from one question to the next.

After completing the questionnaire, the application program transitions back to a display of the home page and changes a shading of the moon icon to indicate that the sleep cycle is on, as shown in FIG. 4E. Subsequent user selection of the moon icon turns the sleep cycle off, to indicate to the application program that the user is no longer sleeping.

The application program also monitors biometrics measured by the computing device or a wearable device in wireless communication with the computing device, to determine that the user has woken up (e.g., due to acceleration movement exceeding a threshold level or being indicative of walking, and/or a gyroscope indicating a standing orientation). In response to such a determination, the computing device may display a dialog box indicating that the user has potentially woken up and prompting the user to turn the sleep cycle on or off, as shown in FIG. 4D. Passage of a certain duration of time without user interaction with the prompt, while the user is still active according to accelerometer and/or gyroscope measurements, can cause the application program to automatically turn the sleep cycle off.

### Assessment

User selection of the chat bubble icon on the home page (or other pages in which the chat bubble icon may be displayed) causes the application program to navigate to an Assessment portion of the application interface. The computing device may also monitor a length of time since the user last interacted with the Assessment portion of the user interface, and upon a threshold amount of time passing (e.g., two weeks), the computing device may issue an operating system wide notification or an in-application dialog box prompting the user to re-take the Assessment.

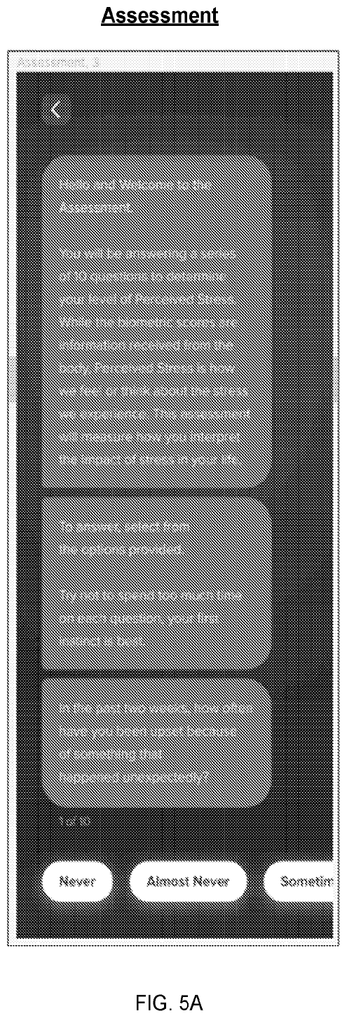

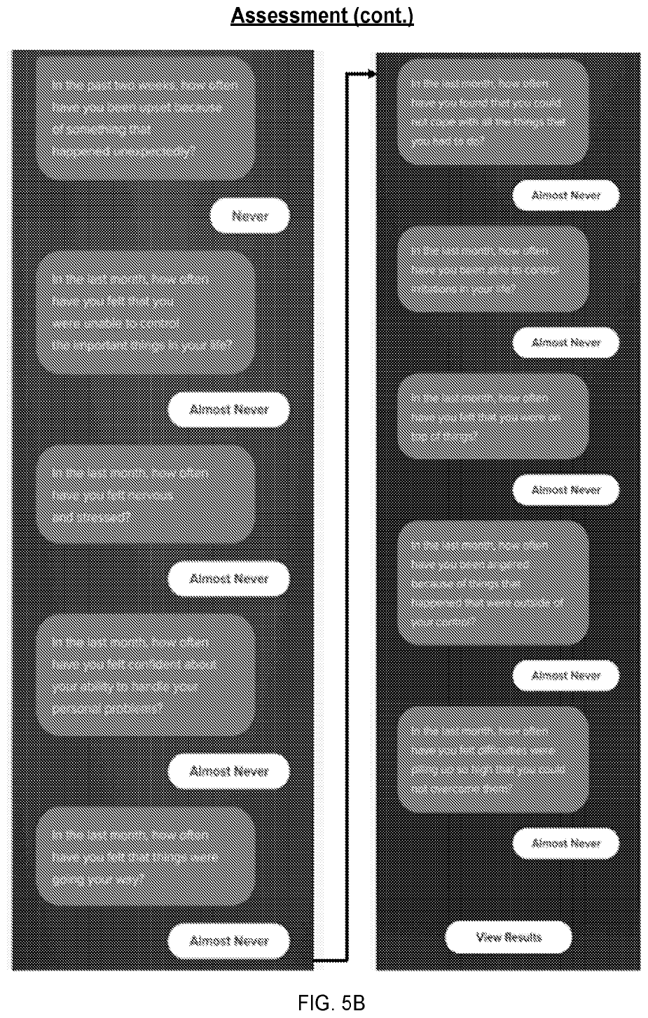

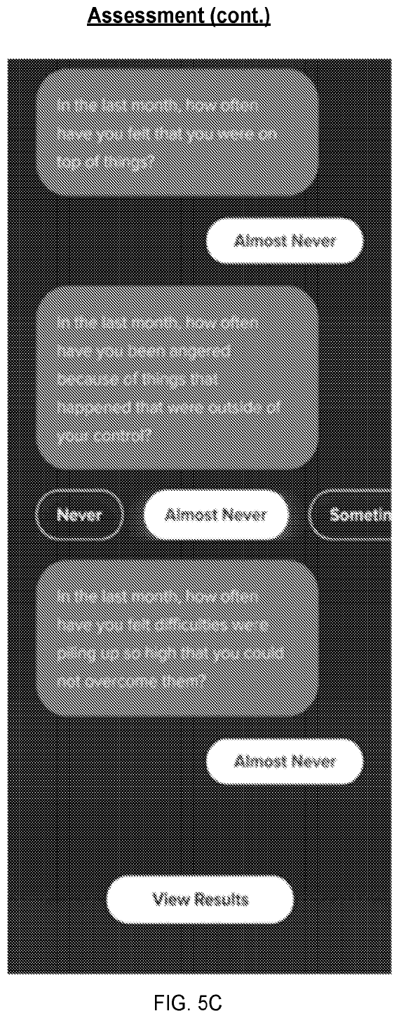

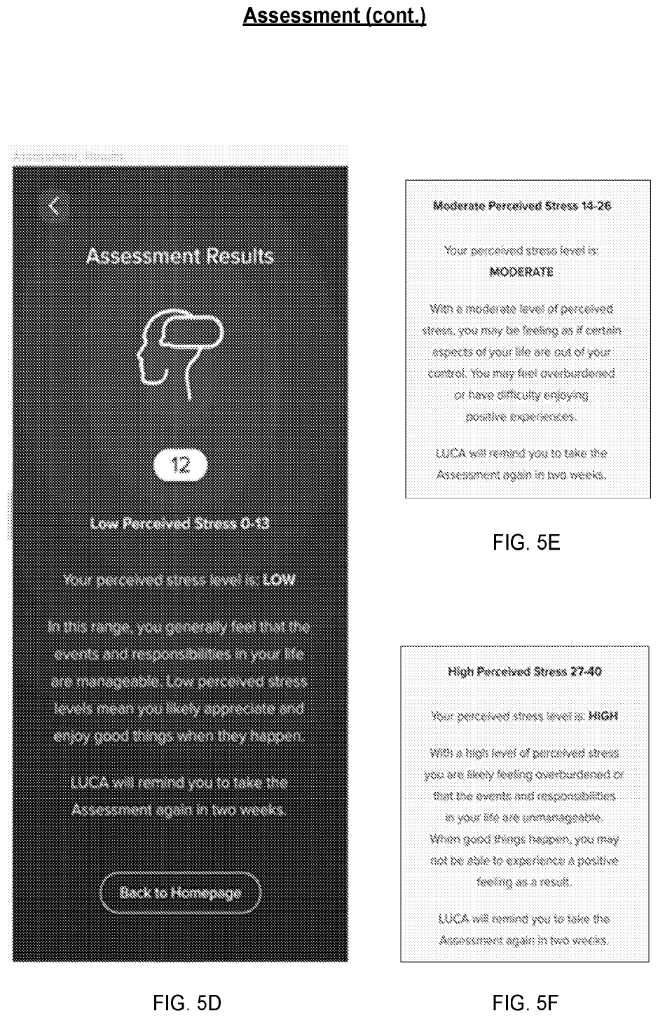

The Assessment portion of the interface presents comments and questions in a chat-style interface in which each comment or question is presented in a user interface chat bubble, with two comments and the first question shown in the interface screen shown in the FIG. 5A user interface.

The first question includes text that prompts the user to answer how often that user has become upset. The application program displays, below the first question interface element, multiple user selectable elements that correspond to different potential answers, including: Never, Almost Never, Sometimes, Fairly Often, and Very Often.

As shown in the FIG. 5A interface screen, only two and a half of these selectable answer interface elements are displayed on the screen at given time. User input may contact a region of the display at which the buttons are displayed and swipe left to cause some of the interface elements that were “off screen” to come onto the screen. Swiping the other direction may cause an inverse removal of some selectable interface elements to return of those previously shown.

A short tap at a displayed location of an interface element: (1) selects the interface element, (2) causes non-selected user interface elements to disappear from the display, and (3) causes the next question to appear on the display along with a corresponding set of selectable user interface elements for answering that question.

The FIG. 5B user interface shows an example chat-style interface in its entirety after the application program has sequentially displayed each question and received an answer thereto. When displayed on a computing device, only a portion corresponding to a currently-scrolled portion of the entire user interface may show at a time.

A user may select an already-answered “answer” interface element to change the answer to the corresponding question. Specifically, in response to user selection of an answer interface element, the application may replace the interface element with the entire set of multiple different, scrollable candidate answer interface elements, as shown in the FIG. 5C interface.

The application program can analyze the user-supplied answers, compare the answers to stored data, rules, and/or functions that map different answers to different levels of stress, generate a perceived stress value, and present the assessment results screen of FIG. 5D to present a perceived stress numerical value. The computing system also identifies a category into which the numerical value falls from the following three categories: “Low”, “Moderate”, or “High”. Content of the moderate and high category screens is shown in FIGS. 5E and 5F.

### Biometrics

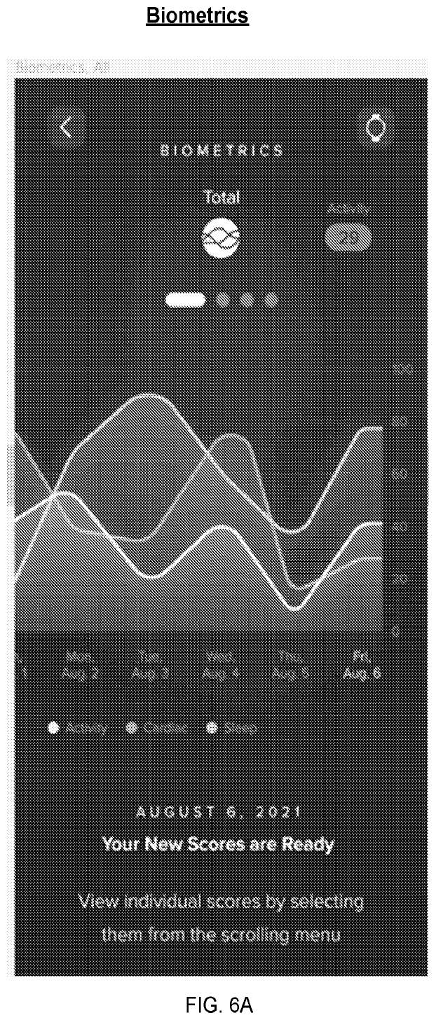

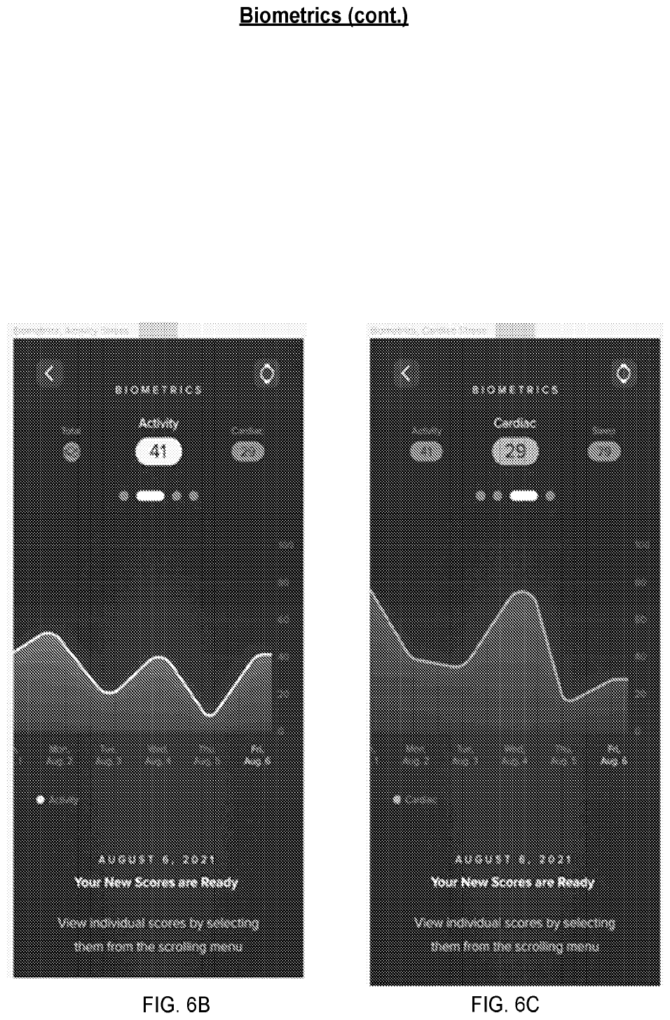

User selection of the centered, circle interface element on the home page causes the application program to navigate to a biometrics portion of the application program, shown in FIG. 6A. This portion of the application program includes various screens with which a user may interact to view metrics that the application program determines based on sensor data received from the computing device or a wearable computing device in communication therewith (e.g., a smartwatch in wireless communication with the computing device via Bluetooth or through the internet).

The biometrics interface screen shows three graph lines that indicate user scores for different biometric values, including “Activity”, “Cardiac”, and “Sleep” scores, determination of each score described below.

User input may swipe to the side at the top of the interface (e.g., where the interface states “Total” in the middle of the display) to cause the interface to transition to a new “filtered” interface screen, in which a graph for only a single metric is displayed. For example, the filtered “Activity” and “Cardiac” screens are shown in FIGS. 6B and 6C.

Each of these three biometric scores can change over time, as shown by the lines varying in the graph, among a dynamic range of values between 0 and 100. The value for each biometric may be calculated based on multiple different inputs (potentially weighted differently), and may be updated regularly (e.g., every minutes, 10 minutes, hour, or day).

### Activity Score Metric

The activity score may be calculated based on two sub-scores that are weighted in calculating the activity score (e.g., 50/50 percent, 30/70 percent).

A first activity sub-score represents daily activity, and may be determined based upon multiple different various inputs. A first example input is minutes of user activity a day that exceeds a threshold. User activity may be determined by a computing system (e.g., the computing device, a cloud system in communication therewith, a wearable device in communication therewith), and may be determined based on analysis of accelerometer and gyroscope data. A second example input is an amount of metabolic equivalents (METs) exhausted by the user in a day. METs represent an amount of energy used by a user during physical activity. The amount of METs per day may be calculated by a computing system based on various biometric sensor inputs, such as analysis of accelerometer data, heart rate data, and/or gyroscope data.

Various combinations of the two example inputs maybe assigned different activity scores by a computer-stored table or computer-executed equation. For example, 80 minutes of qualifying activity in combination with 7 METs that day may correlate to an activity score of high (e.g., 66-100) for the day. As another example 10 minutes of qualifying activity a day in combination with 2 METs that day may correlate to an activity score of low (e.g., 0-33) for the day. As such, more activity and more METs correlate to a higher score, while lower activity and lower METs correlate to a lower score.

### Cardiac Score Metric

The cardiac score may be calculated based on three sub-scores that are weighted in calculating the overall cardiac score (e.g., 33/33/33 percent, 25/25/50 percent).

A first cardiac sub-score is based on an amount of METs exhausted by the user during a sub-portion of the day. For example, a computing system may monitor when METs are exhausted by the user, and calculate an amount of METs exhausted during a certain time period (e.g., from waking up to going to sleep, or from 8 am to 6 pm). Different MET levels may correlate to different values for this first cardiac sub-score. For example, 4 METs may correlate to a sub-score of 10, while, while 7 METs may correlate to a sub-score of 50.

A second cardiac sub-score is based on a heart rate of the user during qualifying activity. Qualify activity is activity that meets a certain threshold of intensity (e.g., activity determined by a computing system to represent exercising, based on analysis of an increasing heart rate and/or intensity of accelerometer data). The heart rate of the user during qualifying activity may be compared to a target heart rate for the user, which the computing system can identify based on any combination of an age, height, weight, or a body-mass index (BMI). For example, for a thirty-year old person, a heart rate that is 30% below the target heart rate may correlate to a sub-score of 40, a heart rate that is 10% below the target heart rate may correlate to a sub-score of 80, and a heart rate that is over the target heart rate may correlate to a sub-score of 100.

A third cardiac sub-score is based on weekly averages of the first cardiac sub-score and the second cardiac sub-score. For example, a weekly average of 6 METs per day and an average weekly heart rate during qualifying activities that is 20% below the target heart rate may correlate to a sub-score of 60.

### Sleep Score Metric

The sleep score may be calculated based on nine sub-scores that are weighted in calculating the overall sleep score (e.g., with the same or different weights).

A first sleep sub-score is based on an amount of time that it takes a user to fall asleep. This amount of time may be triggered based on the user pressing the “moon” icon from the home screen of the application program. The amount of time may correspond to the time that the user selecting the moon icon, or may correspond to the time that the user completes the questions that are presented responsive to the user selecting the moon icon. A computing system may then measure an amount of time that passes before a determination is made by a computing system that the user has fallen asleep, for example, based on a drop in heart rate and/or user movement measured by an accelerometer falling below a threshold (e.g., at an instant or an average over a certain amount of passed time). A computing system may correlate a time-to-sleep of 2 minutes to a sub-score of 80. A time-to-sleep of 40 minutes may correlate to a sub-score of 20.

A second sleep sub-score is based on daily sleep efficiency. For example, a computing system may calculate a total amount of time the user spends in bed trying to sleep, starting based on a time based on the user selects the moon icon and continuing. The total amount of time the user specs in bed trying to sleep may extend until a computing system determines that the user is oriented upward and walking (e.g., based on analysis of gyroscope and/or accelerometer data) and/or receiving user selection of an interface element indicating that the sleep cycle is to turn off.

The application program may turn the sleep cycle off if the displayed prompt to turn on or off the sleep cycle is presented by the computing device for a threshold period of time without the application receiving user input, should the user be determined to be walking around and not laying down trying to sleep. The time at which the system determines that sleep ends may correlate to the time that the user is determined to have stood up and or moved more than a threshold amount, even if the user confirms a few minutes later by selection of an interface element that the user is indeed awake.

An amount of time that the user is asleep, during the period that the user is determined to be in bed, may be determined to be an amount of time while the user is laying down trying to sleep in which analysis of biometric data indicates that the user is sleeping, in distinction to being in bed and tossing and turning while being awake and trying to sleep. For example, the wearable device may make a determination regarding whether the user is asleep and if so, a level of depth to that sleep. For example, the wearable device may send to the application program an indication that the user is in one of the following states: Awake, Light sleep, Deep sleep, or REM. Times in which the user is determined to be in any of the light sleep, deep sleep, or REM states during the period in which the user is determined to be trying to sleep can count towards time that the user is asleep.

The application program can calculate a proportion of amount of time that the user is determined to be asleep to a total amount of time that the user is determined to be in bed. A computing system may then correlate the calculated proportion to a sub-score based on stored information, a stored table, stored rules, and/or stored equations. For example, a sleep efficiency of below 80% may correlate to a sub-score of 45. A sleep efficiency of greater than 90% may correlate to a sub-score of 80.

A third sleep sub-score can represent a weekly sleep efficiency, and can be based on a combination of (i) an average sleep efficiency during the week, and (ii) an average hours asleep per day over the week. For example, an average sleep efficiency of 83% and average hours asleep/day of 7.5 can correlate to a sub-score of 85.

A fourth sleep sub-score can represent a level of sleep fragmentation during a given night. For example, the application program can receive from the wearable device an indication every time the user is determined to be no longer sleeping. The application program can identify a number of times that the user is determined to be trying to sleep but is not sleeping that exceeds a certain length of time before the user falls back asleep (e.g., 3 minutes). A computing system can correlate this identified number of times to a sub-score. For example, waking 3times for more than 10 minutes can correlate to a sub-score of 20. Waking 1 time for more than 10 minute can correlate to a sub-score of 75.

A fifth sleep sub-score can represent an extent to which the user awakens early. For example, a computing system can identify a number of times during the last week (or an average of a number of times over multiple weeks) that the user wakes up before a given time, such as 4:30 am. The computing system can correlate this number of times awakening early to a sub-score based on various information/rules/equations. For example, waking before 4:30 am twice in a week can correlate to a sub-score of 15. Waking before 4:30 am no times during a week can correlate to a sub-score of 100.

A sixth sleep sub-score can represent a weekly sleep deficit. Inputs used in determining the weekly sleep deficit can include an average weekly sleep efficiency, and an average weekly sleep debt in hours. The average weekly sleep debt in hours may be calculated based on a total amount of time the user is actually asleep, in comparison to a target sleep time. A computing system may calculate the target sleep time based on any combination of the user's age, weight, BMI, etc. For example, a computing system may correlate a weekly sleep efficiency of 87% and an average weekly sleep debt of 3 hours of sleep debt/week averaged over multiple weeks to a sub-score of 65.

A seventh sleep sub-score can represent a nocturnal heart rate. For example, a computing system may calculate an average heart rate of the user while asleep (e.g., an entire time laying down, or time in which the user is determined to actually be sleeping while laying down). A computing system may then correlate this average heart rate to a sub-score. For example, an average nocturnal heart rate of 73 may correlate to a sub-score of 85. An average nocturnal heart rate of 85 may correlate to a sub-score of 45.

An eighth sleep sub-score can represent a cardiac dipping point. For example, a computing system may calculate the average heart rate of the user during the day (e.g., an average of a few hours before the user selected the sleep icon). A computing system may then determine an amount that the user's heart rate dipped during sleep, where the dip is measured down to an absolute minimum heart rate during sleep, or to the average nocturnal heart rate. The system can correlate a dip of 15% to a sub-score of 15, and a dip of 25% to a sub-score of 60.

A ninth sleep sub-score can represent a total sleep time deviation from a target sleep level. For example, a computing system can identify a number of days that the user deviates more than a threshold amount from a target sleep time, as established for the user by a computing system based on demographic information for the user (e.g., age, sex, weight, height).

### Stress Score Metric

The computing system may correlate each sub-score for the above-described three biometric measurements (Activity, Cardiac, and Sleep) with an absolute stress value (e.g., 2, 5, or 6) or a relative stress value (e.g., 0, −3, or +3), and may combine the stress values from all sub-scores into a final stress score for a certain time period (e.g., for the day). Generally, the lower the scores calculated for any of the Activity, Cardiac, and Sleep scores and sub-scores, the greater likelihood the stress score will be high. Conversely, higher scores for the Activity, Cardiac, and Sleep scores and sub-scores, the greater likelihood the stress score will be low. A computing system may then correlate the stress score to one of multiple stress categories, for example, “Low”, “Medium”, and “High”, and can graphically indicate the current stress category on the homepage (e.g., in the middle of the circle interface element).

### Odyssey Interactive Experience

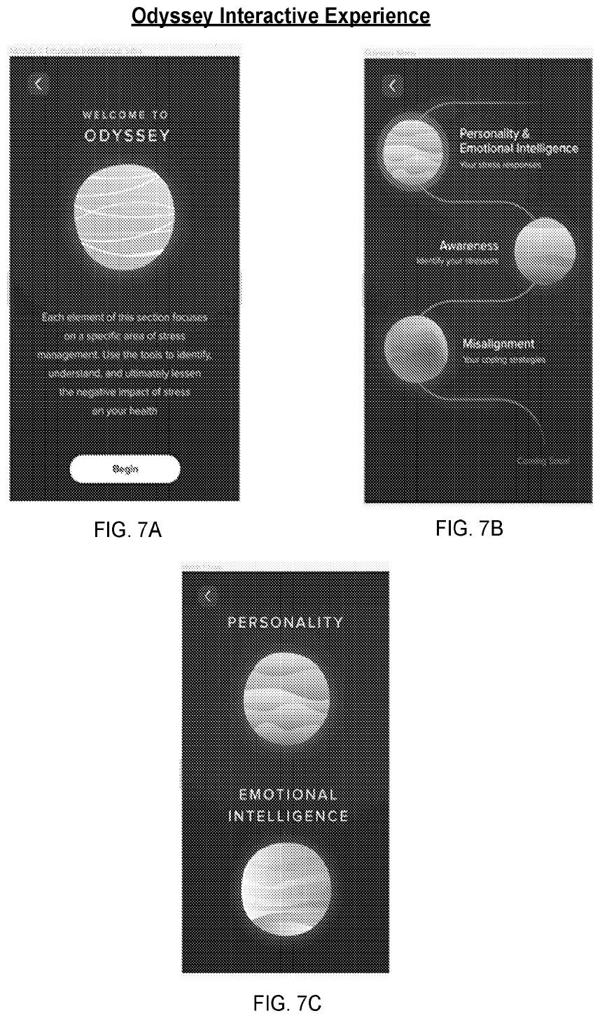

User selection of an interface element to launch an interactive experience, labelled “Odyssey” in the depicted example, at the top of the FIG. 3 home page interface causes the application program to transition to an “Odyssey” portion of the application program, which provides a human-computer interactive experience that assists the user in managing their stress. The application program may show the FIG. 7A user interface the first time that the user access the Odyssey portion of the application. Each subsequent navigation to the Odyssey portion may start with the Odyssey menu user interface, which is shown in FIG. 7B.

The Odyssey menu presents three selectable interface elements for three respective modules, to assist users in managing their stress:
(1) A “Personality and Emotional Intelligence” module to identify user characteristics and how the user responds to stress;
(2) An “Awareness” module to identify the user's stressors; and
(3) A “Misalignment” module to determine how the user copes with their identified stressors.

User selection of the interface element for “Personality and Emotional Intelligence” causes the application program to navigate to a sub-menu shown by the FIG. 7C interface. This sub-menu enables the user to select either the “Personality” sub-module or the “Emotional Intelligence” sub-module. Selection of each element (e.g., a fast touchscreen tap) causes navigation to a screen for the corresponding sub-module.

### Personality Sub-Module

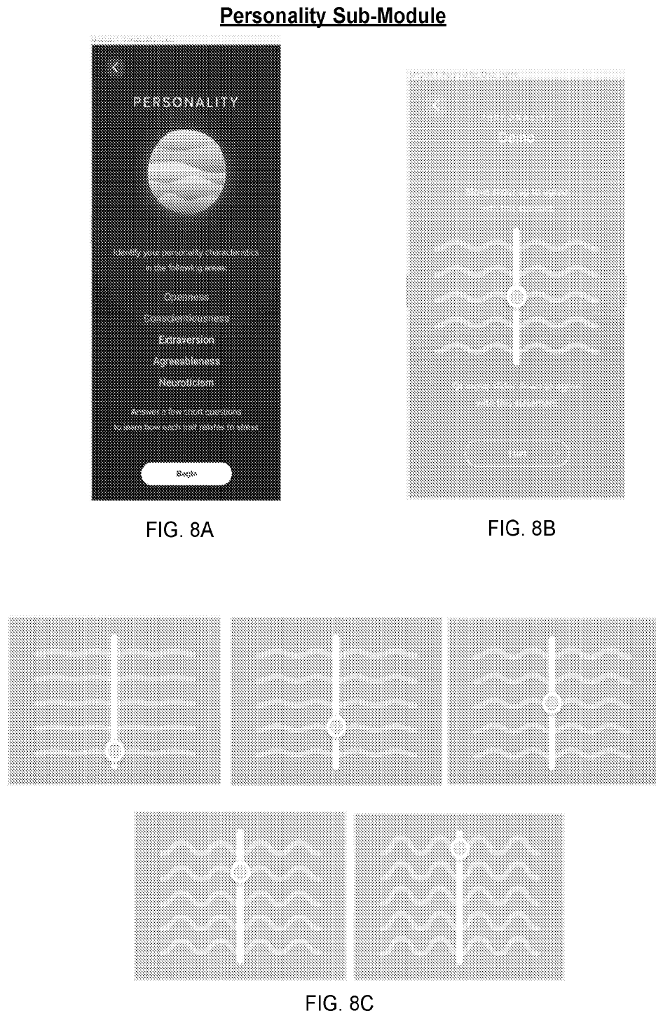

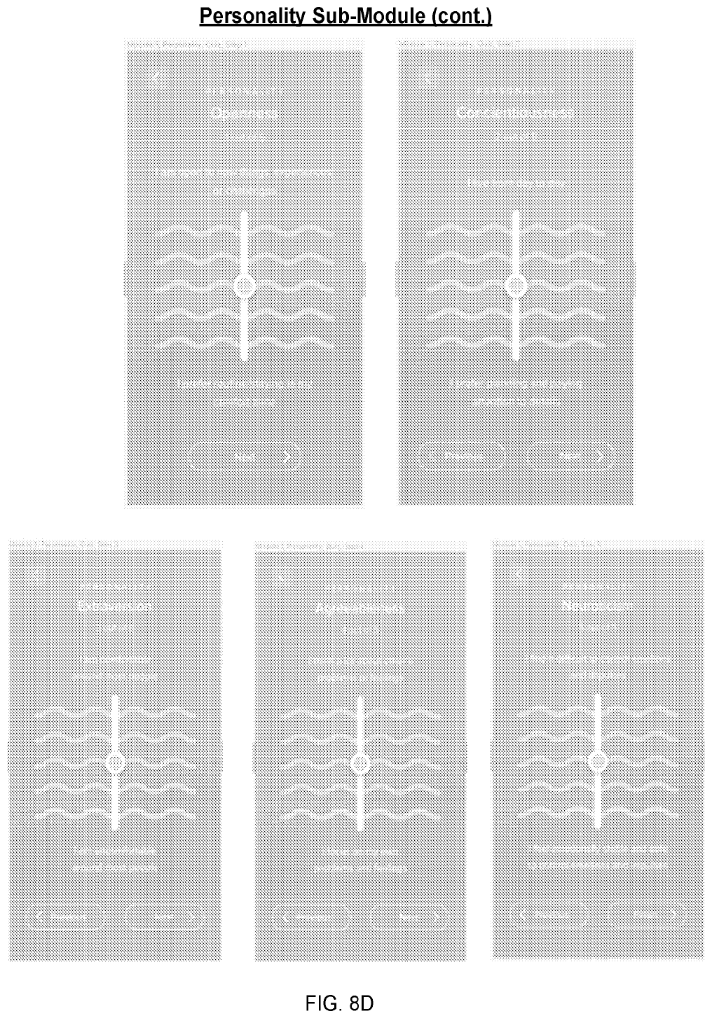

FIG. 8A shows an introductory screen that the computing device presents upon user selection of the above-referenced “Personality” interface element. FIG. 8B shows a demonstration interface screen presented after the user select the “Begin” interface element.

The demonstration interface screens include a vertical bar, over which the computing device presents a user-slideable control element (e.g., slideable in response to a user input touching the control element and moving the user input up or down across the touchscreen), which can advantageously permit the user to input accurate amounts using non-numeric values. Responsive to user-modification of the position of the control element, the five lines shown behind the vertical bar change from wavy to relatively straight, representing calm or turbulent water. Different positions of the control element correspond to different patterns for the five lines in the background, as illustrated in FIG. 8C.

The user can select the start interface element in the FIG. 8B screen to cause the computing device to step-wise present five prompts on five different interface screens, shown in FIG. 8D. The user can interact with the slideable control element presented as part of each screen, to indicate with which of the two statements presented as part of the prompt on each screen the user agrees most (or an intermediate position indicating a neutral opinion or leaning one way or another). Sliding the control element toward the bottom statement indicates a user preference generally for calm/steadiness/introversion, while sliding the control element toward the top statement indicates a user preference generally for change/extroversion.

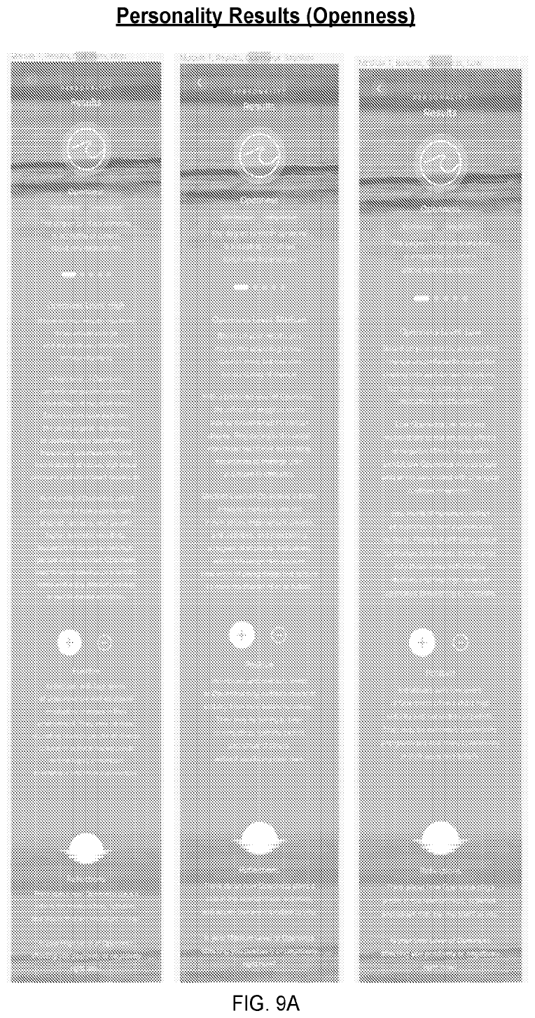

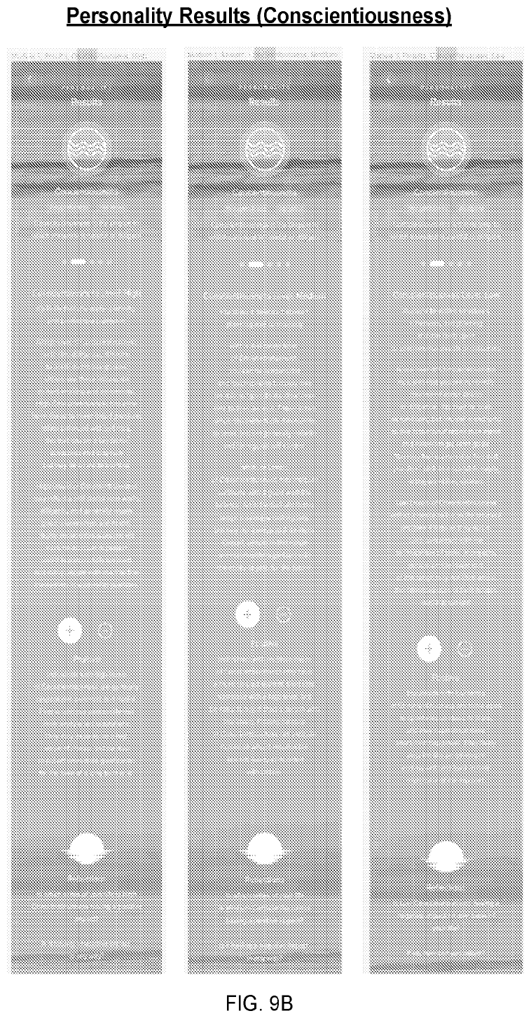

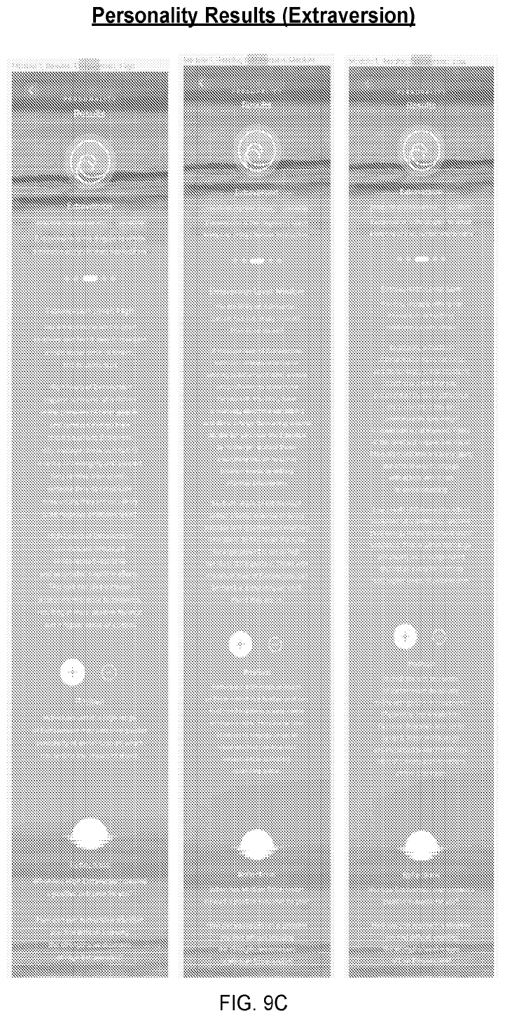

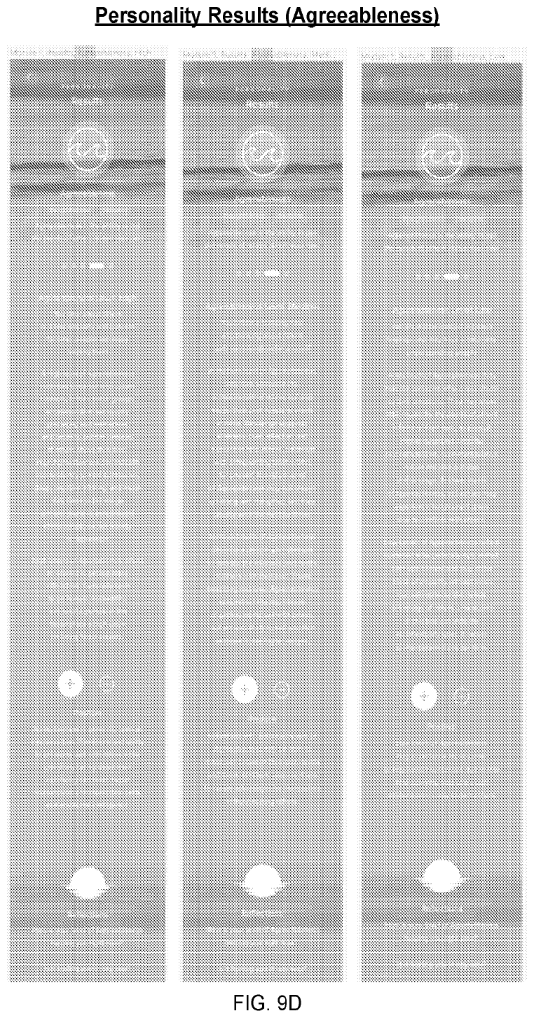

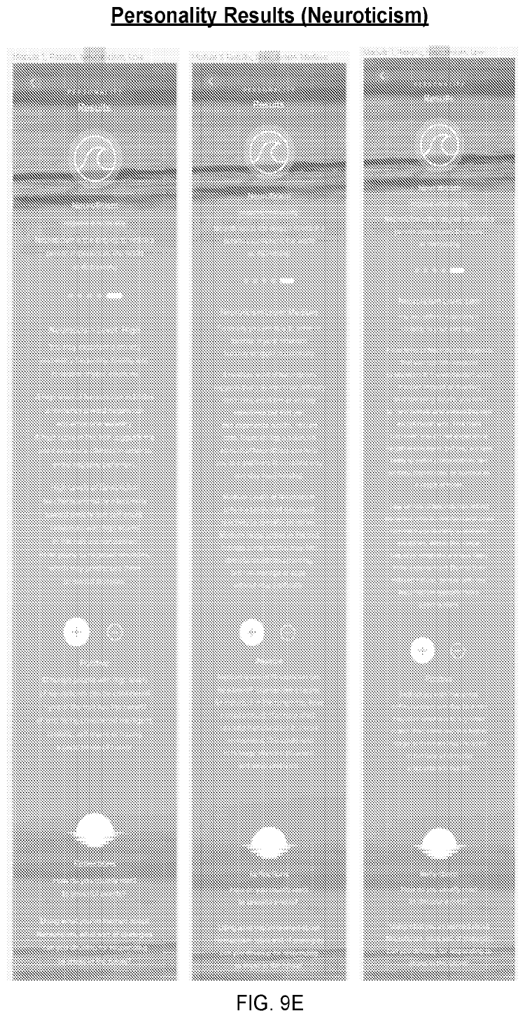

Responsive to the user providing input at each of the above-described screens, the computing system provides five response screens that each indicate the user's tendencies in each of five personality traits: Openness (see FIG. 9A screens), Conscientiousness (see FIG. 9B screens), Extraversion (see FIG. 9C screens), Agreeableness (see FIG. 9D screens, and Neuroticism (see FIG. 9E screens). The result for each trait is selected by the application program based on the user input for the corresponding prompt shown in FIG. 8D.

### Emotional Intelligence Sub-Module

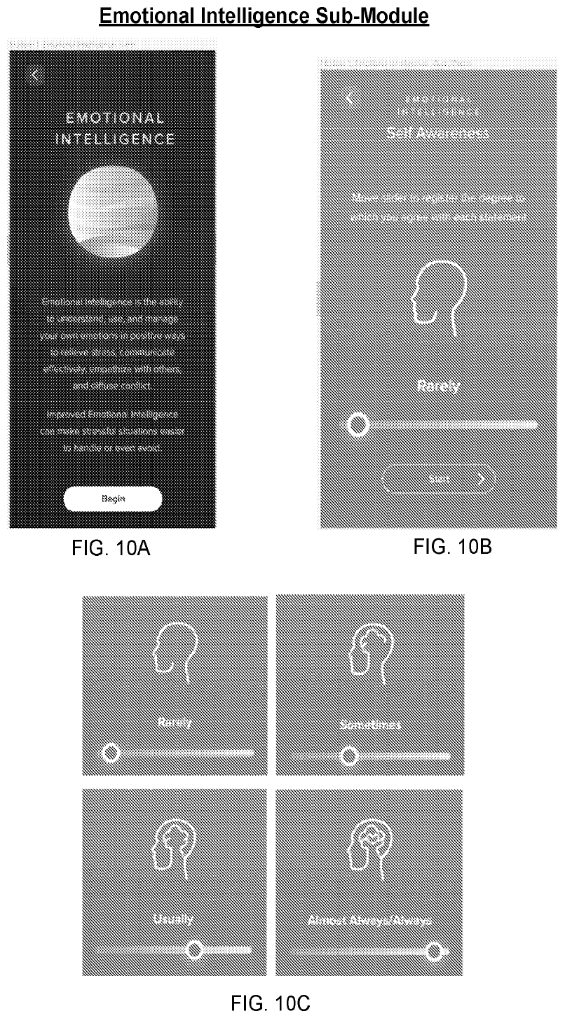

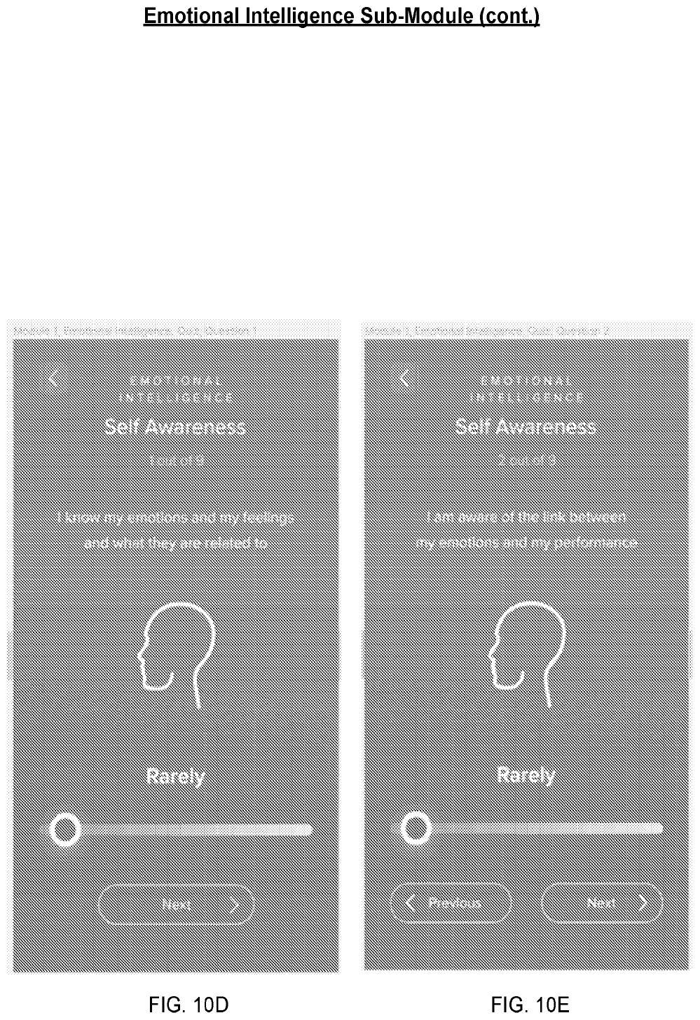

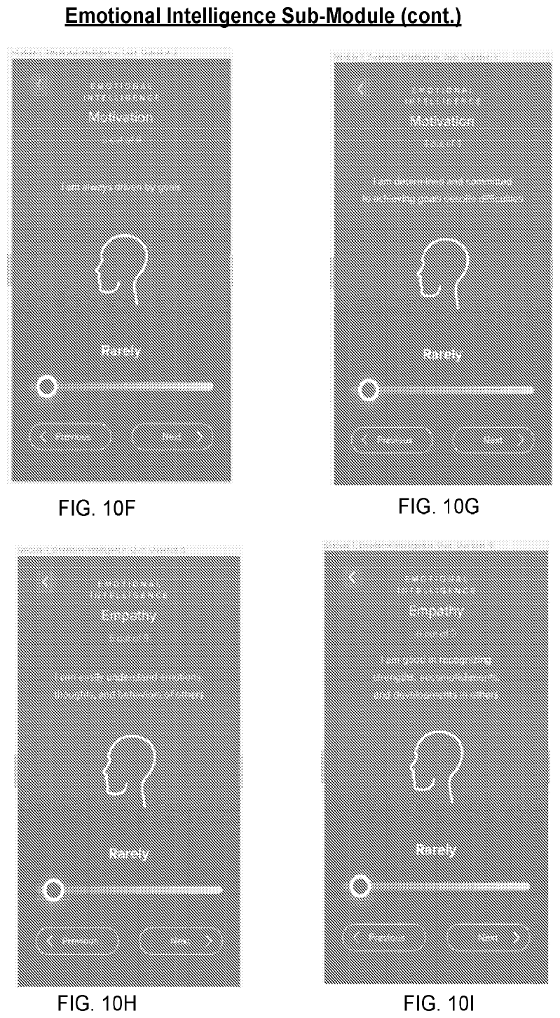

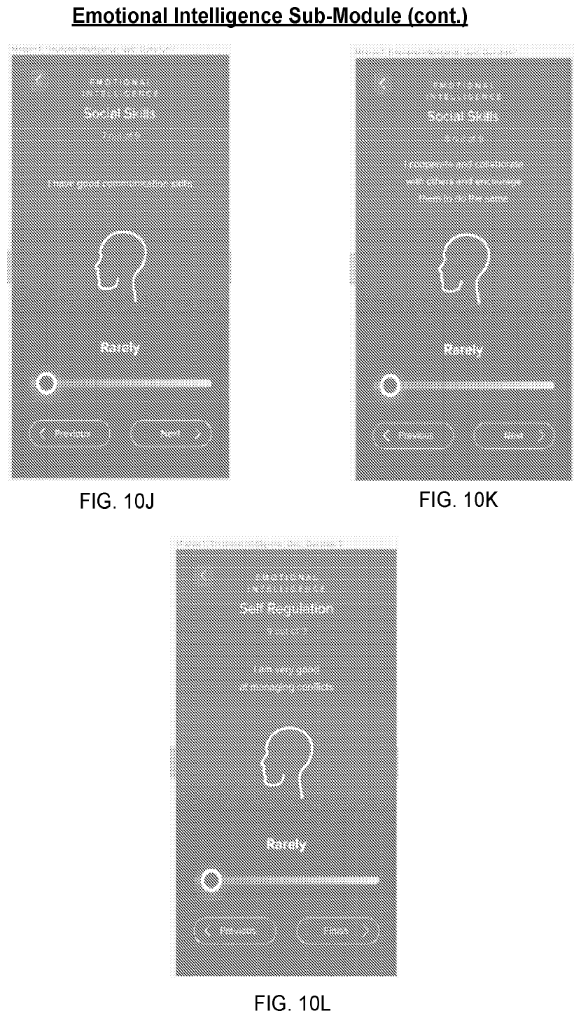

The FIG. 10A screen is an introductory screen that the computing device presents upon user selection of the “Emotional Intelligence” interface element in the FIG. 7C interface. FIG. 10B shows a demonstration interface screen that is presented after the user selects the “Begin” interface element in the FIG. 10A interface.

The demonstration interface screen includes a horizontal bar, over which the computing device presents a user-slideable control element (e.g., slideable in response to user input contacting the control element and sliding left or down across the touchscreen). Here again, such a user-interface feature can advantageously permit the user to input accurate magnitudes for each response while using non-numeric values. Responsive to user-modification of the position of the control element, the figure of a human head shown above the horizontal bar changes from less complex to more complex, representing the following four candidate answers: Rarely, Sometimes, Usually, Almost Always/Always. These different levels of detail in the figure are illustrated by FIG. 10C.

The user can select the start interface element to cause the computing device to step-wise present nine statements on nine respective interface screens, illustrated in FIGS. 10D-L The user can interact with the control element presented as part of each screen to indicate an answer to each statement. Sliding the control element toward the right indicates the user being more agreeable with the statement. In response, the illustration of a human head changes to be more-filled-out to provide visual feedback to the user regarding whether the user does not think much about the identified topic or does think much about the identified topic.

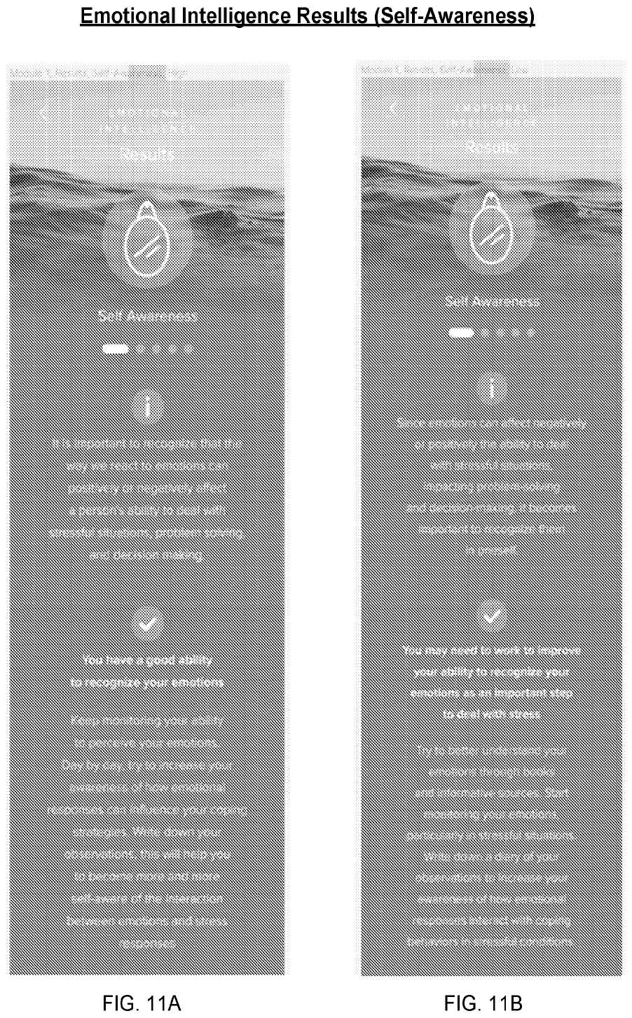

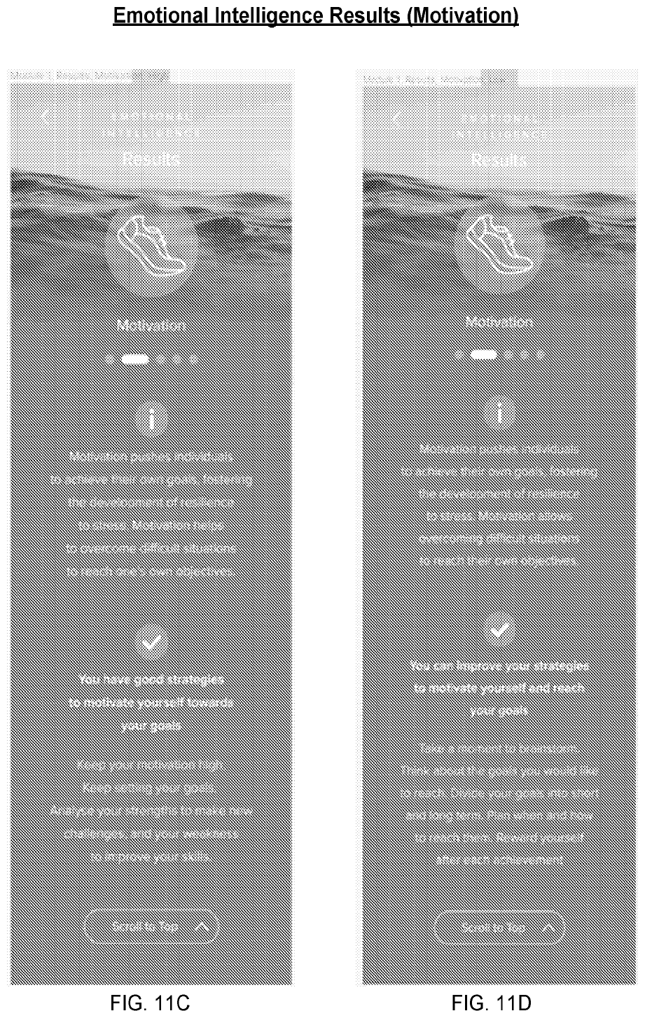

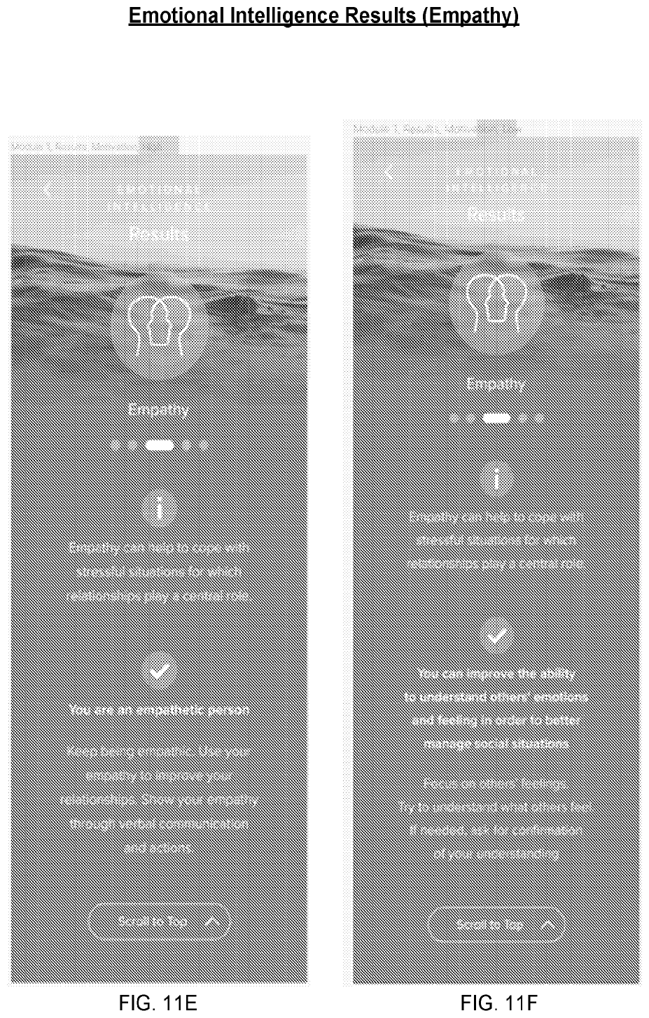

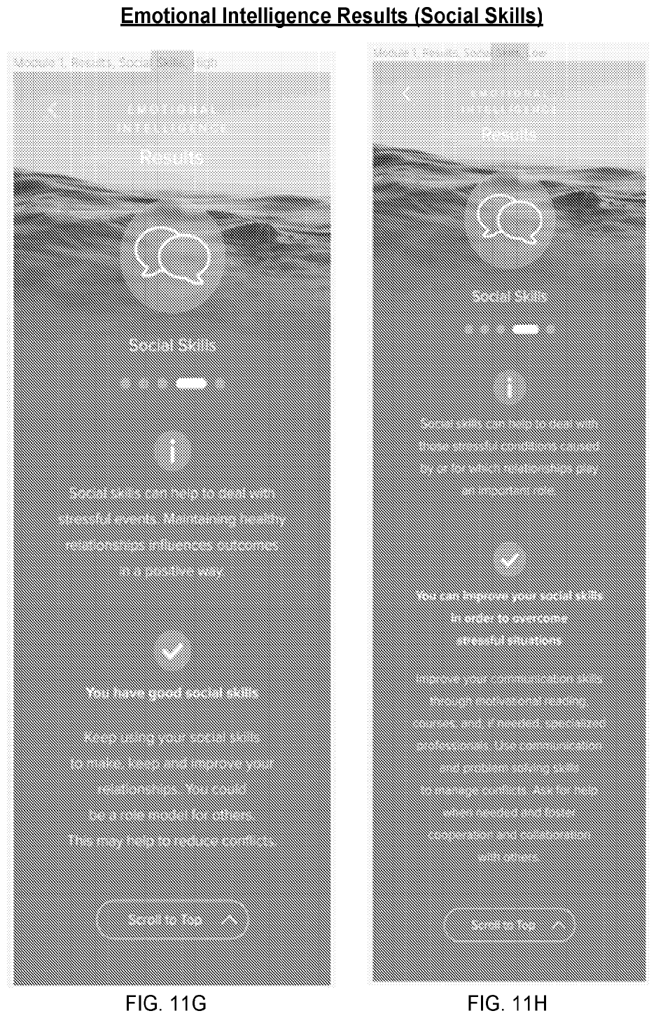

Responsive to the user providing input at each of the above-described screens, the computing system provides nine response screens that each indicate the user's tendencies in each of five emotional intelligence traits: Self-Awareness (see FIGS. 11A-B), Motivation (see FIGS. 11C-D), Empathy (see FIGS. 11E-F), Social Skills (see FIGS. 11G-H), and Self-Regulation (see FIGS. 11I-J). The result for each trait is selected by the application program based on the user input provided for the questions shown in FIGS. 10D-L.

### Awareness Module

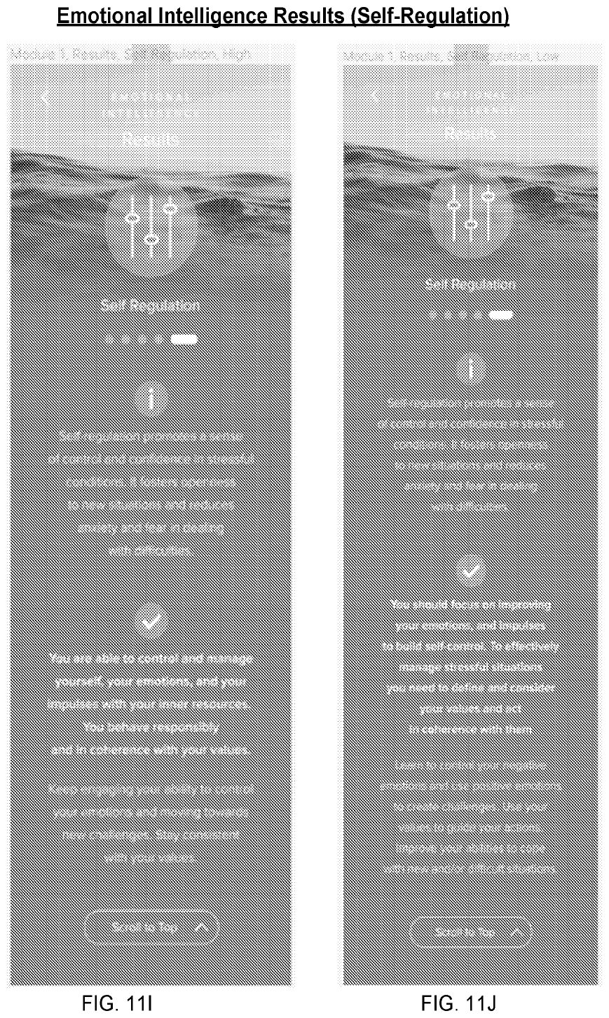

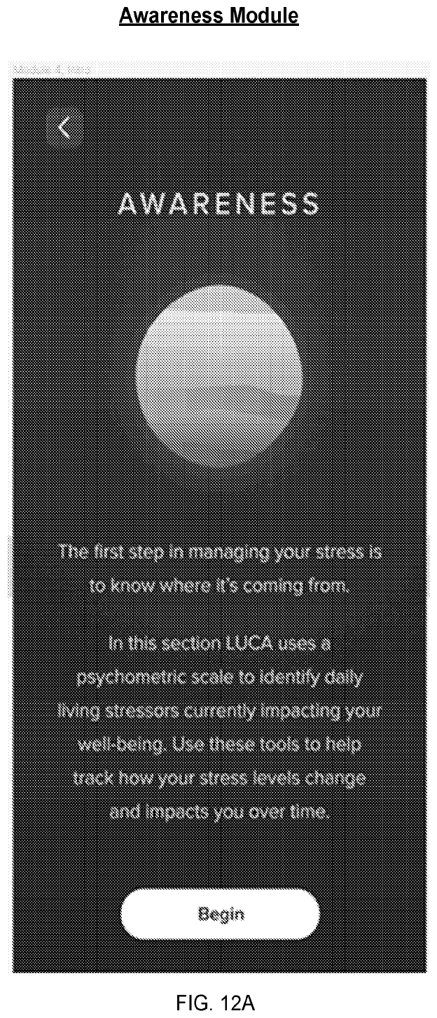

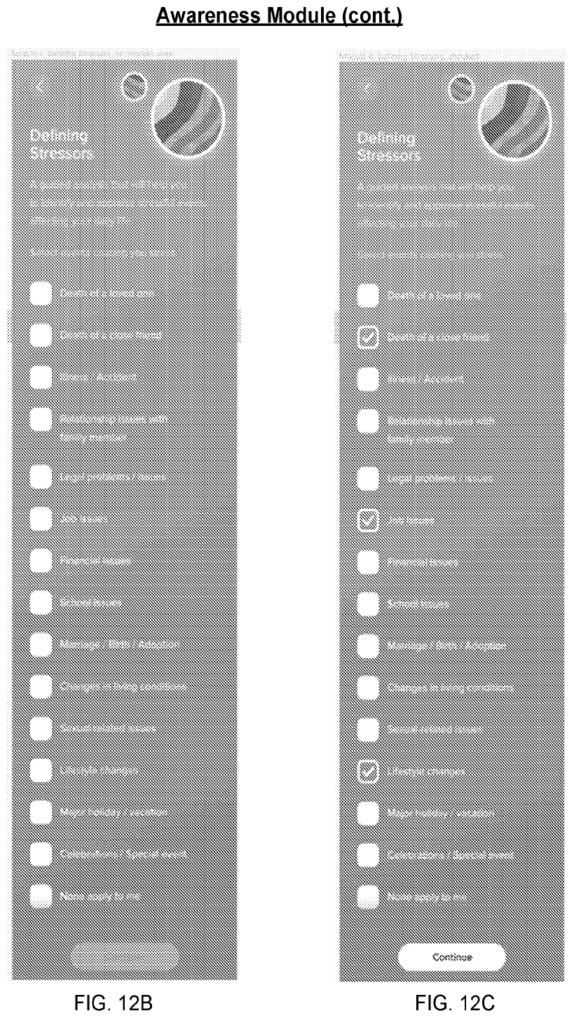

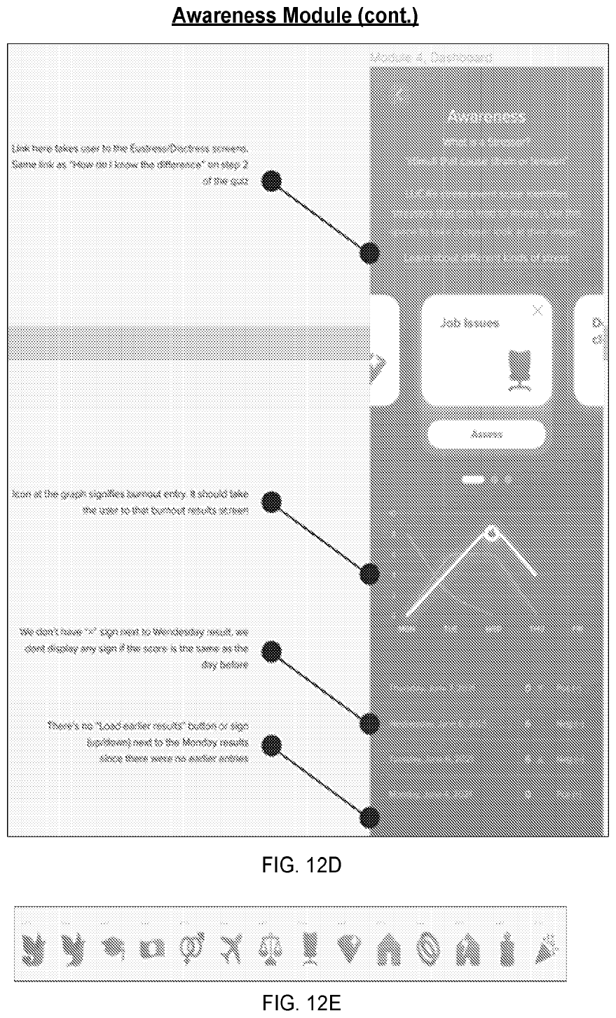

FIG. 12A shows an introductory screen that the computing device presents upon user selection of the “Awareness” interface element from the FIG. 7B Odyssey main menu.

After user input selects the “Begin” interface element, the application program transitions to a screen, illustrated in FIG. 12B, that includes multiple user-selectable elements that the user may select to specify which stressors the user is experiencing.

Responsive to user selection of zero or more stressors (e.g., three selections in the FIG. 12C interface) and selecting the “Continue” interface element, the application program navigates to the Awareness “Dashboard” illustrated in FIG. 12D.

The dashboard provides customized information responsive to the user's selection(s) of particular stressor(s), such as specific content associated with each stressor that the user had selected. In the dashboard, the dashboard is showing content for the “Job Issues” stressor. The FIG. 12D screen presents:
An icon and corresponding text that indicates the presentation of Job Issues content in the current interface screen.
An “Assess” interface element that the user may select to assess the user's stress for this stressor (this changes to state “Reassess” once the user has completed an initial assessment).
A graph with a highlighted/emphasized line that shows current and historical values for this stressor, identified as a result of multiple Assessments/Reassessments in a graphical form (non-emphasized lines are for other stressors).
A table that shows the current and historical values for this stressor in a primarily-textual form.

User input may select at least part of the user interface (e.g., the icon and correspond text) and slide the user input to the left or right to change the user interface to present content for a different stressor. Each stressor from the above-shown selectable list of stressors is associated with an icon from the collection of icons shown in FIG. 12E.

### Distress/Eustress Interface

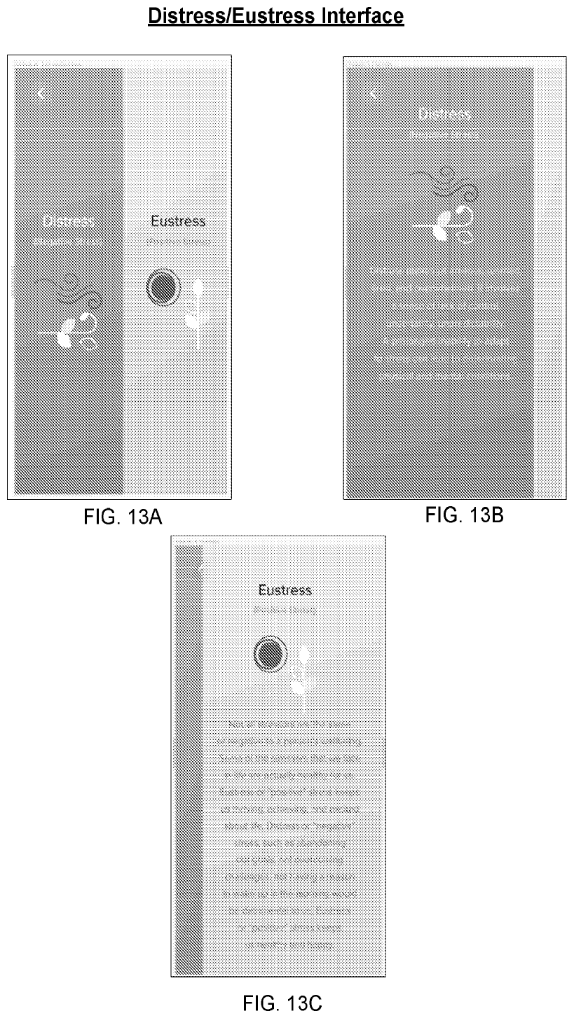

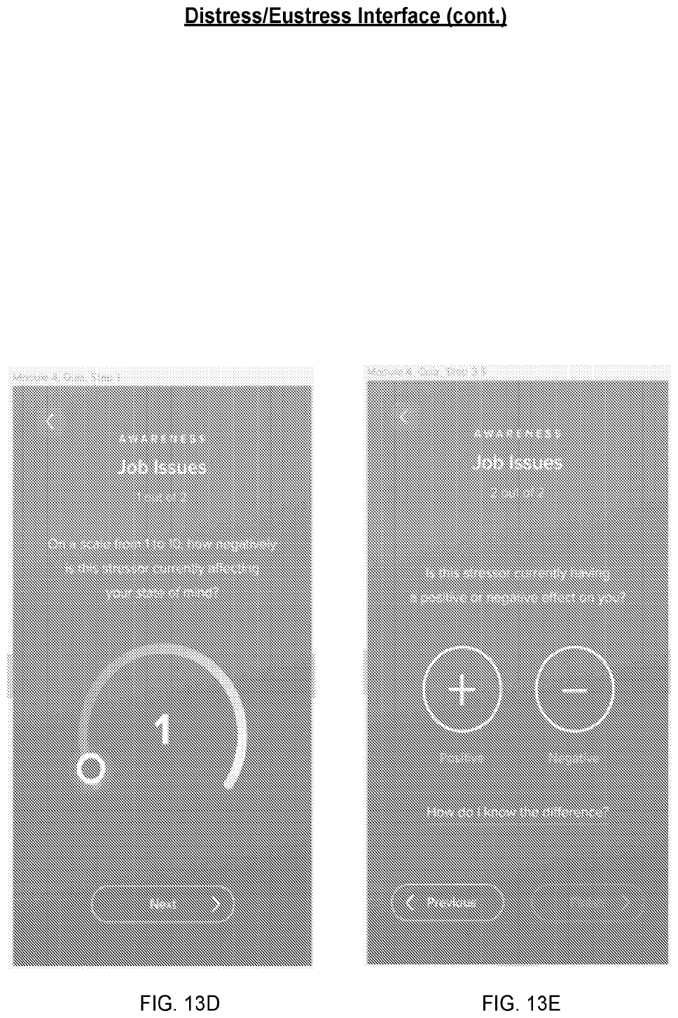

Responsive to user input selecting “Learn about different kinds of stress” in the FIG. 12D Dashboard, the application program transitions to the interface shown in FIG. 13A, which a user can slide to the sides to learn about distress/eustress, as illustrated by FIGS. 13B-C.

Responsive to user input selecting the “Assess” interface element for a certain stressor (see FIG. 12D), the application program presents a sequence of two interactive interface screens, illustrated in FIGS. 13D-E, to determine an extent that the stressor is affecting the user.

Should the user indicate a value for the “Job Issues” stressor that exceeds a certain threshold value (e.g., 7 or greater), for a certain period of time (e.g., a single day, multiple consecutive days, an average of multiple days), the application program may prompt the user to answer multiple questions relating to whether the user is suffering from occupational burnout.

### Occupational Burnout

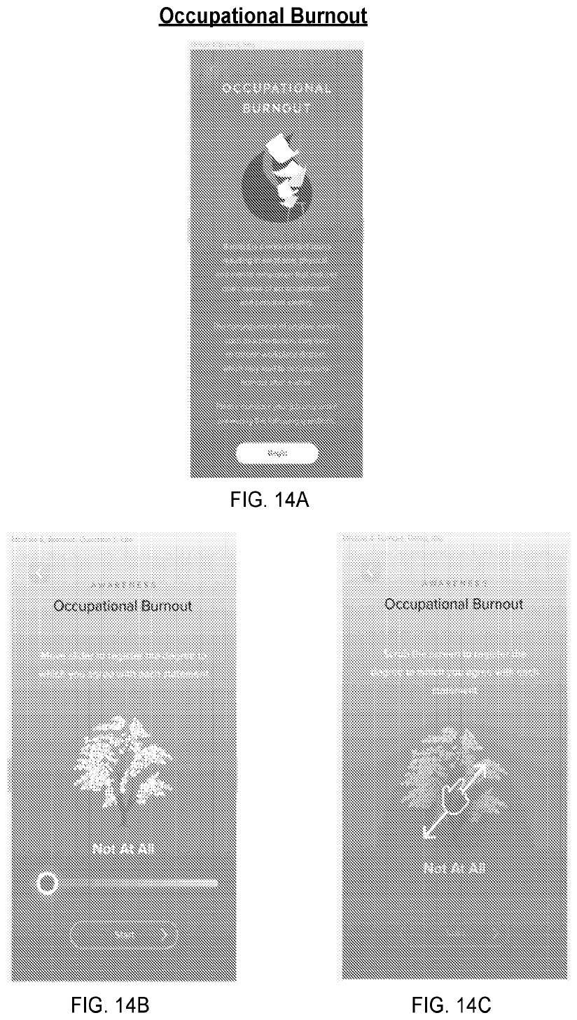

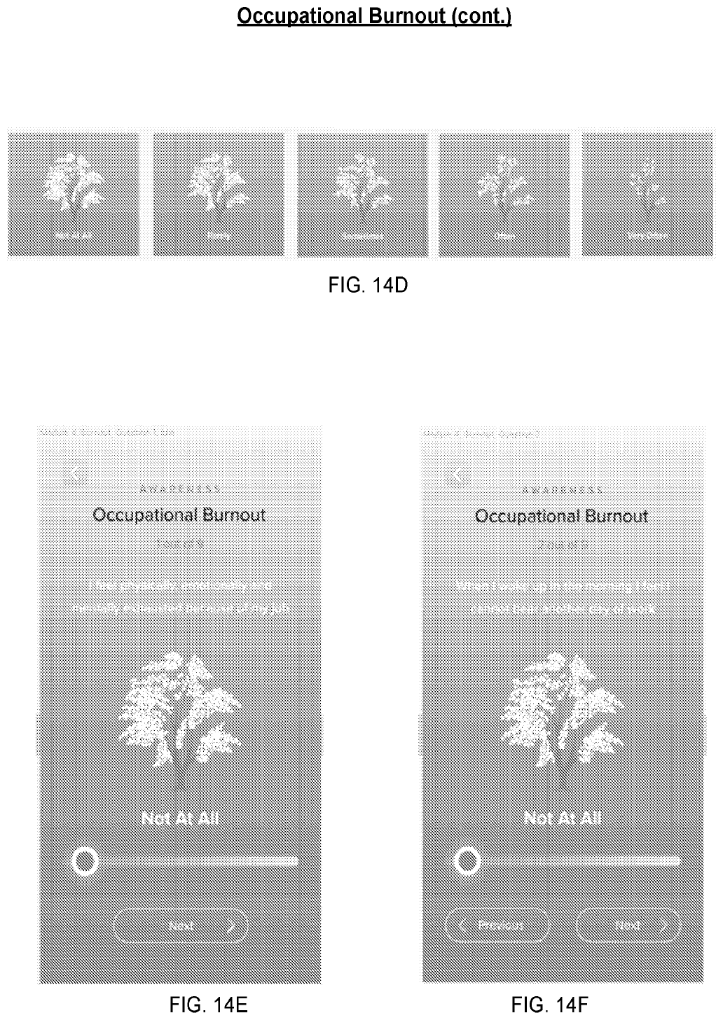

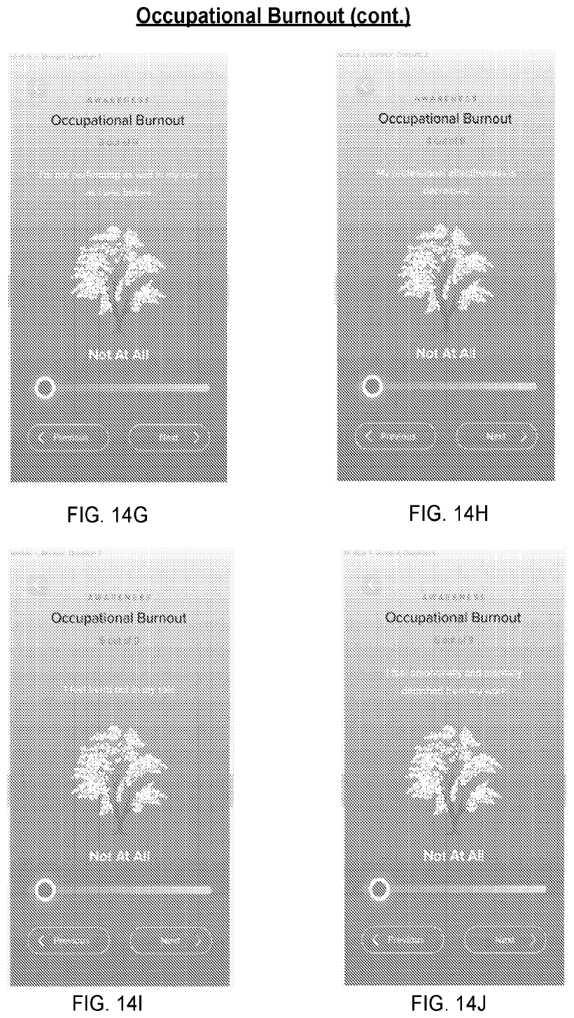

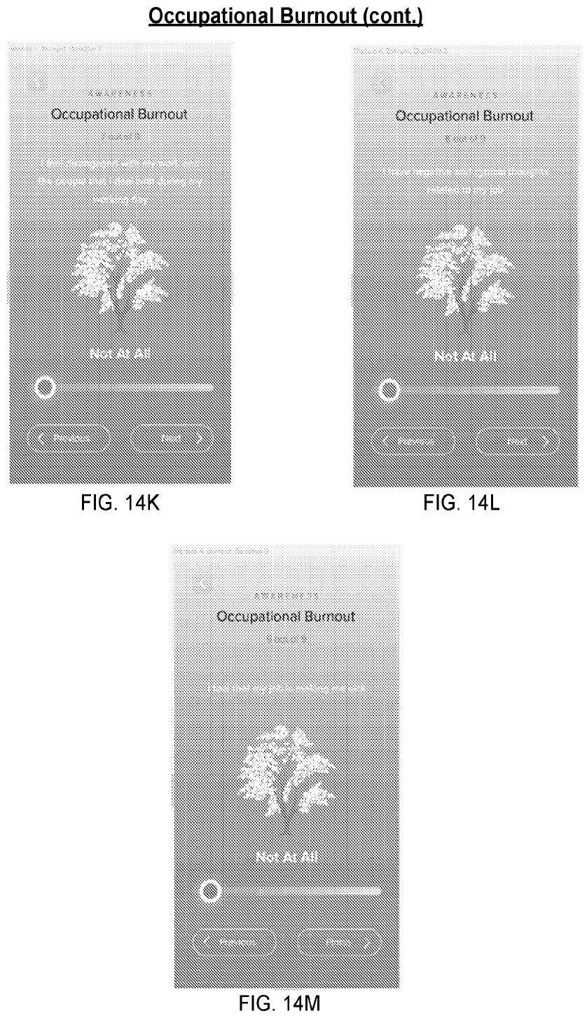

FIG. 14A shows an introductory screen that the computing device presents upon a user beginning the Occupational Burnout portion of the application program.

Multiple manners are envisioned for user input to specify an extent to which the user agrees with a statement presented as part of the occupational burnout portion of the application program. The screen illustrated in FIG. 14B includes a horizontal bar, over which the computing device presents a user-slideable control element. Responsive to user-modification of the position of the control element, the figure of a tree loses or adds leaves to specify one of the following user responses to a statement on the display: Not at all, Rarely, Sometimes, Often, and Very Often. An alternative example mechanism, illustrated in FIG. 14C, does not include a scroll bar, and a user slides their finger directly across the figure of the tree to affect the amount of leaves on the tree and select a user response to the statement on the display. The different versions of the tree figure and associated user response are illustrated in FIG. 14D.

After user input that selects the “Begin” interface element, the application program sequentially presents nine interactive screens, illustrated by FIGS. 14E-M, with which user input may interact to specify one of the above-described answers.

### Burnout Results

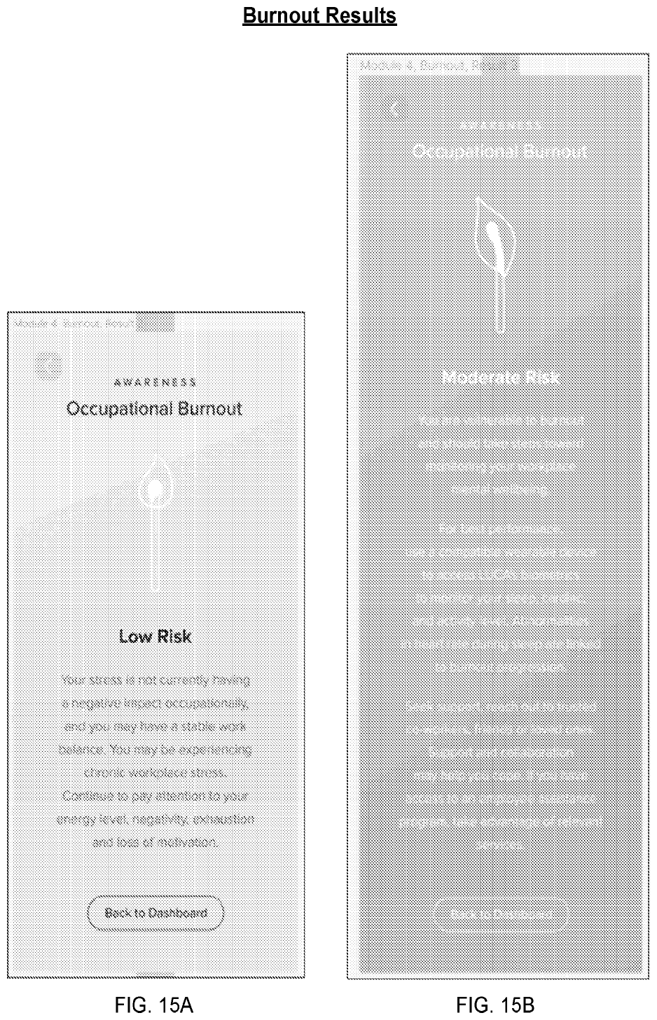

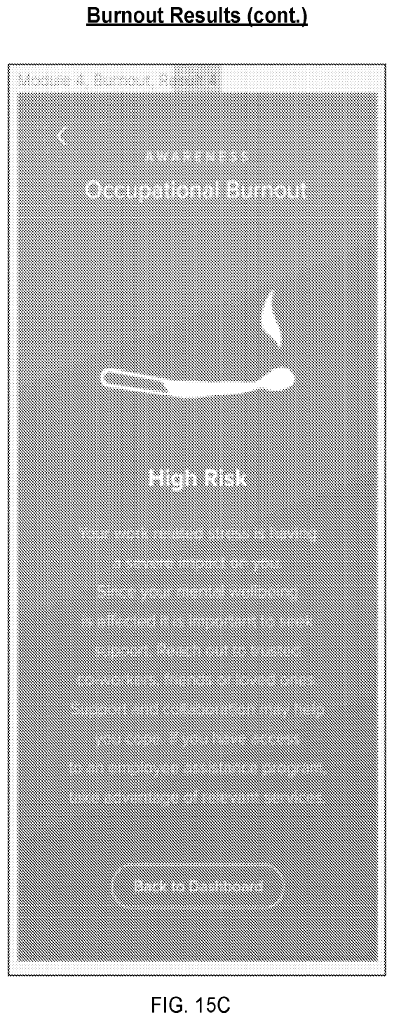

The application program assigns the user one of three burnout levels, illustrated by FIGS. 15A-C, based on the user-selected answers provided through interaction with the interface screens of FIGS. 14E-M.

### Misalignment Module

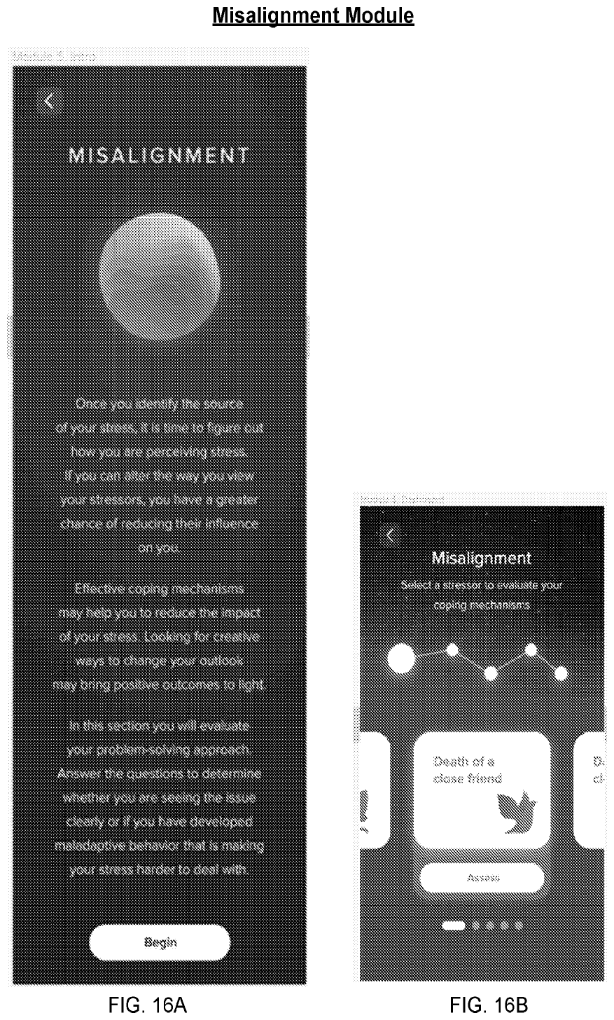

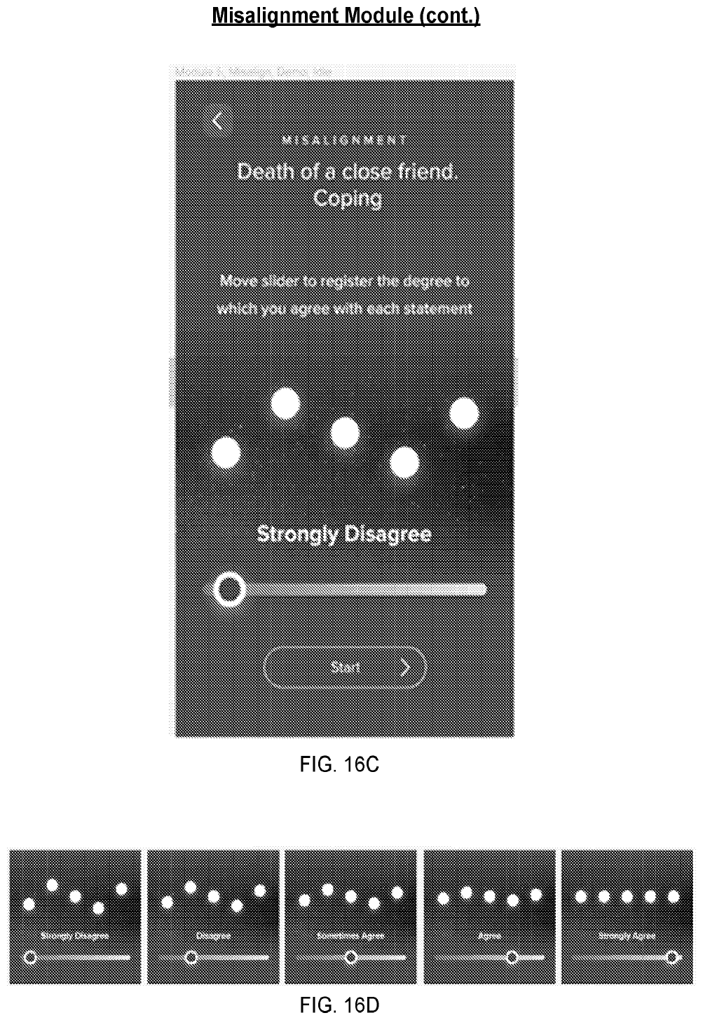

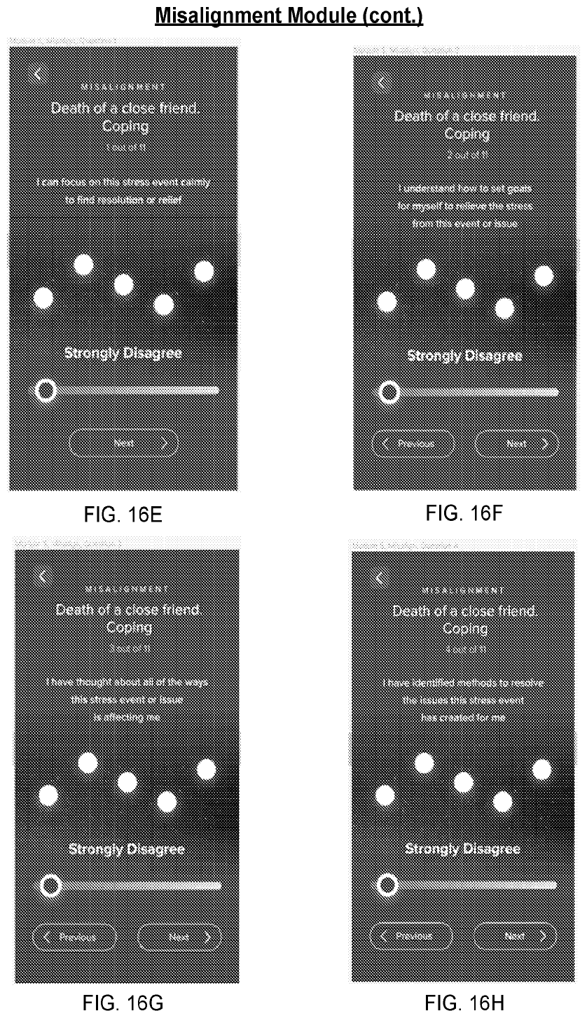

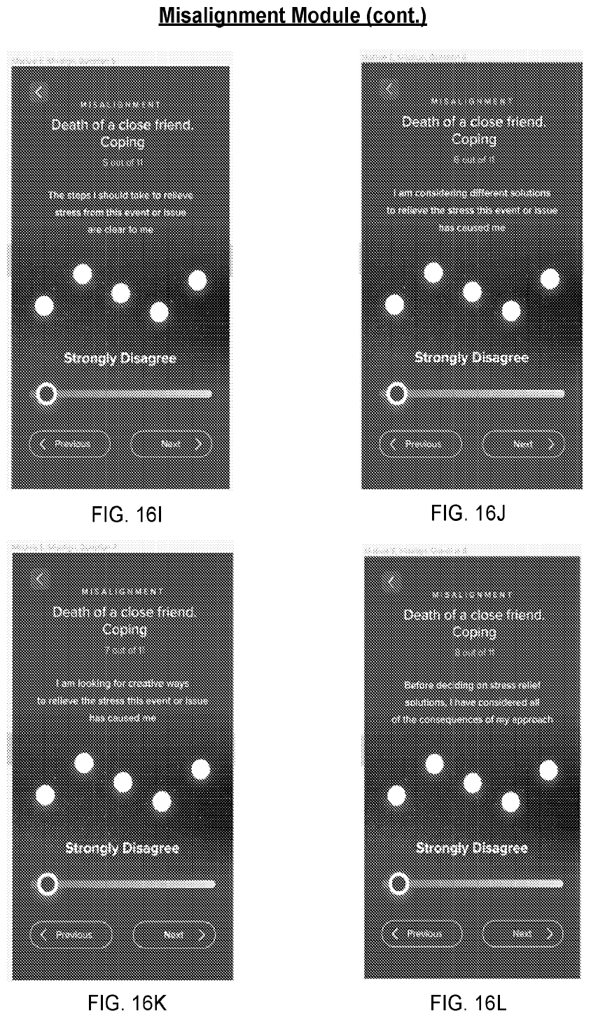

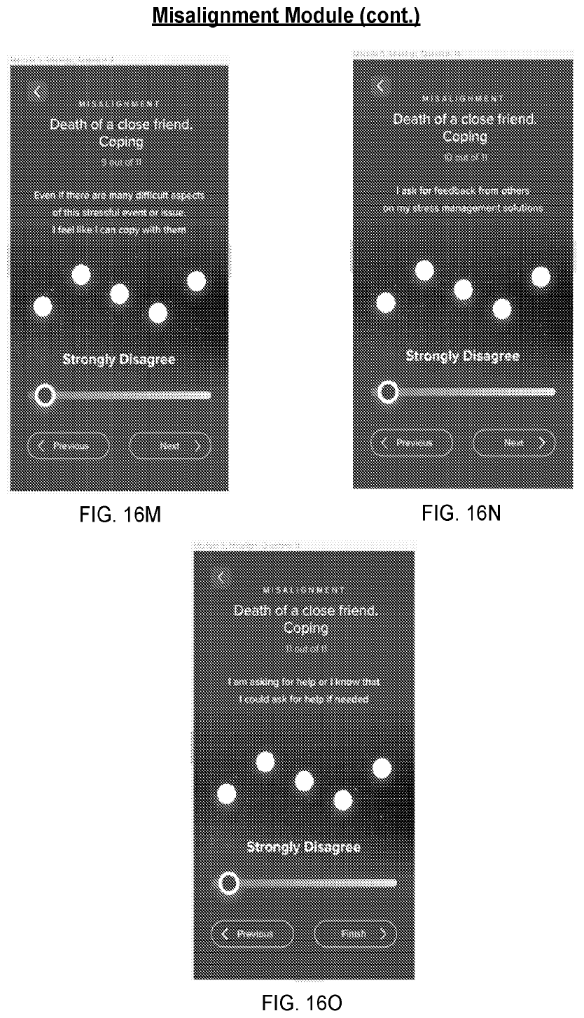

FIG. 16A shows an introductory screen that the computing device presents upon user selection of the “Awareness” interface element from the Odyssey main menu illustrated in FIG. 7B.

After user input selects the “Begin” interface element, the application program transitions to a Misalignment “Dashboard”, illustrated by FIG. 16B. The dashboard includes an icon and descriptive text for each stressor that the user selected when interacting with the “Awareness” portion of the application program, along with an “Assess” button that the user can select to begin an assessment for the corresponding stressor. The “Assess” button changes to display “Reassess” if the user has already performed an assessment for the particular stressor.

User input may select at least part of the user interface (e.g., the icon and correspond text) and slide the user input to the left or right to cause the user interface to graphically rotate/slide to present a content for a different stressor.

After user-selection of the “Assess” or “Reassess” interface element for a particular stressor, the application program transitions to presenting a demonstration screen, illustrated by FIG. 16C, with example interface controls which the user can interact to experience how to answer the soon-to-be-displayed questions. The screen includes a horizontal bar, over which the computing device presents a user-slideable control element. Responsive to user-modification of the position of the control element and as shown in FIG. 16D, the position and/or motion of ball interface elements shown on the display screen changes, and a user-selected answer changes between the following answers: Strongly Disagree, Disagree, Sometimes Agree, Agree, and Strongly Agree.

Responsive to user selection of the “Start” interface element, the application program presents a sequence of screens with which user input may interact to indicate an extent to which the user agrees or disagrees with the statement provided on each respective screen, as illustrated by FIGS. 16E-O. The questions may be the same for each stressor for which the user performs this process.

### Misalignment Results

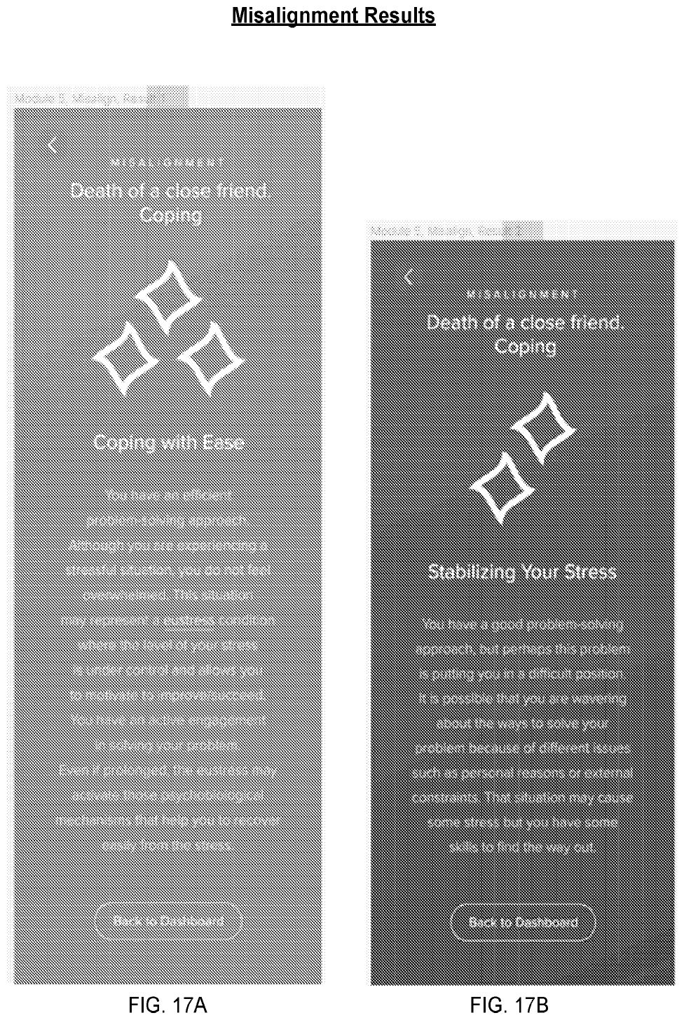

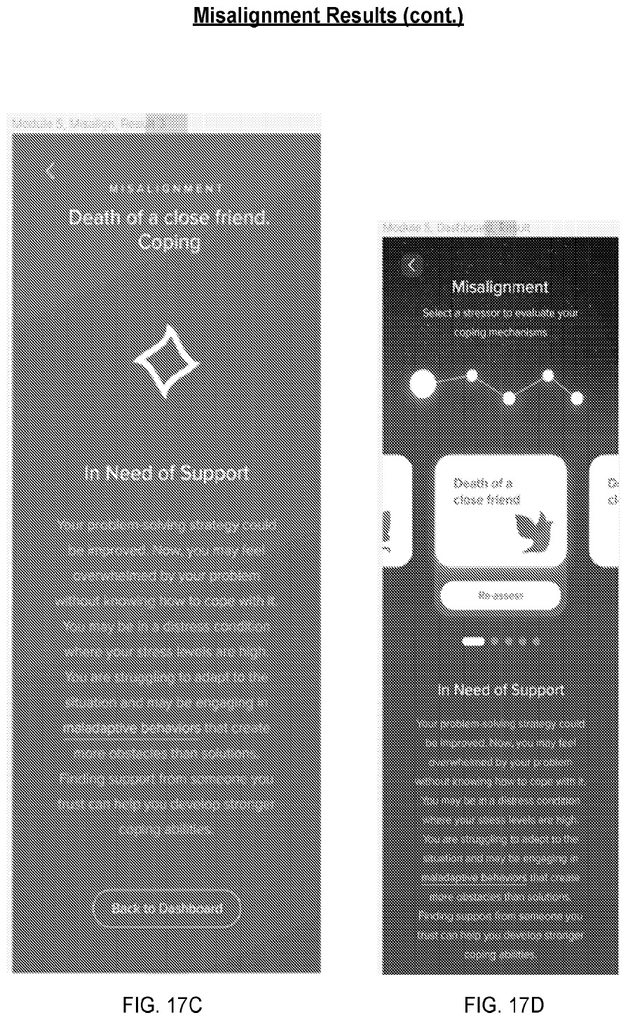

Responsive to user input selecting the “Finish” interface element upon answering all eleven questions, the application program determines a category to describe characteristics of the user's coping behaviors, based on the user's answers to the questions. An example presentation of the interface shown for each of the determined categories is shown by FIGS. 17A-C.

Responsive to user-selection of the “Back to Dashboard” interface element, the application program transitions back to the Misalignment Dashboard, illustrated in FIG. 17D, which the application program updates to specify the category that describes the characteristics of the user's coping behaviors.

### Reminders

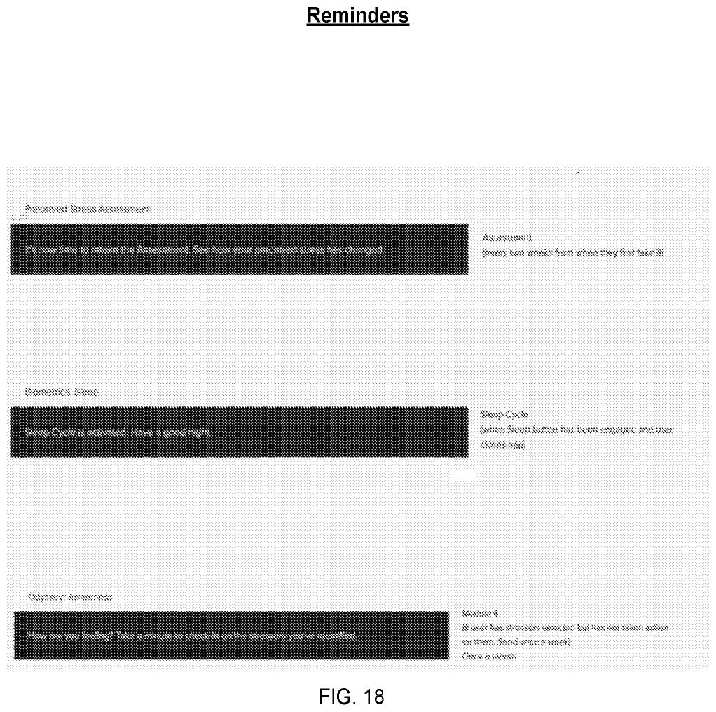

The application program may monitor an amount of time that has passed since the user has interacted with various portions of the application program. Should a threshold amount of time pass since interaction with a various portion of the application program, the application program can trigger the computing device to display a push notification, even if the user is not currently viewing screens of the application program (e.g., to be displayed when the user is viewing a screen of another application program, or the home screen of the computing device operating system). Example notifications are shown by FIG. 18.

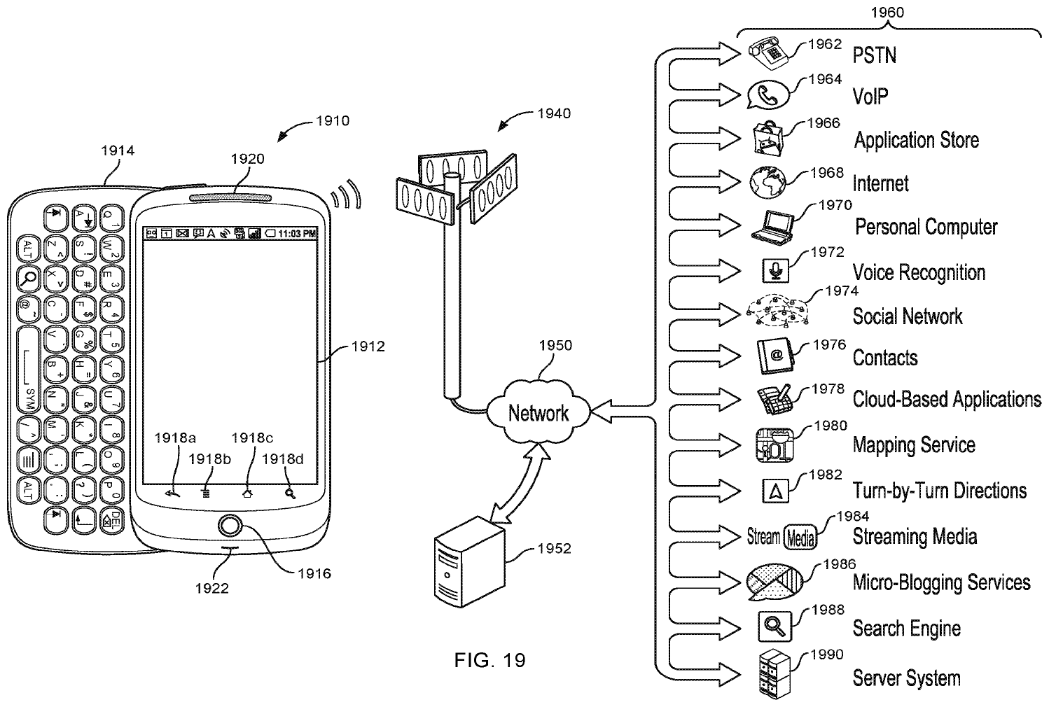

Referring now to FIG. 19, a conceptual diagram of a system that may be used to implement the systems and methods described in this document is illustrated. In the system, mobile computing device 1910 can wirelessly communicate with base station 1940, which can provide the mobile computing device wireless access to numerous hosted services 1960 through a network 1950.

In this illustration, the mobile computing device 1910 is depicted as a handheld mobile telephone (e.g., a smartphone, or an application telephone) that includes a touchscreen display device 1912 for presenting content to a user of the mobile computing device 1910 and receiving touch-based user inputs and/or presence-sensitive user input (e.g., as detected over a surface of the computing device using radar detectors mounted in the mobile computing device 510). Other visual, tactile, and auditory output components may also be provided (e.g., LED lights, a vibrating mechanism for tactile output, or a speaker for providing tonal, voice-generated, or recorded output), as may various different input components (e.g., keyboard 1914, physical buttons, trackballs, accelerometers, gyroscopes, and magnetometers).

Example visual output mechanism in the form of display device 1912 may take the form of a display with resistive or capacitive touch capabilities. The display device may be for displaying video, graphics, images, and text, and for coordinating user touch input locations with the location of displayed information so that the device 1910 can associate user contact at a location of a displayed item with the item. The mobile computing device 1910 may also take alternative forms, including as a laptop computer, a tablet or slate computer, a personal digital assistant, an embedded system (e.g., a car navigation system), a desktop personal computer, or a computerized workstation.

An example mechanism for receiving user-input includes keyboard 1914, which may be a full qwerty keyboard or a traditional keypad that includes keys for the digits ‘0-9’, ‘*’, and ‘#.’ The keyboard 1914 receives input when a user physically contacts or depresses a keyboard key. User manipulation of a trackball 1916 or interaction with a track pad enables the user to supply directional and rate of movement information to the mobile computing device 1910 (e.g., to manipulate a position of a cursor on the display device 1912).

The mobile computing device 1910 may be able to determine a position of physical contact with the touchscreen display device 1912 (e.g., a position of contact by a finger or a stylus). Using the touchscreen 1912, various “virtual” input mechanisms may be produced, where a user interacts with a graphical user interface element depicted on the touchscreen 1912 by contacting the graphical user interface element. An example of a “virtual” input mechanism is a “software keyboard,” where a keyboard is displayed on the touchscreen and a user selects keys by pressing a region of the touchscreen 1912 that corresponds to each key.

The mobile computing device 1910 may include mechanical or touch sensitive buttons 1918a-d. Additionally, the mobile computing device may include buttons for adjusting volume output by the one or more speakers 1920, and a button for turning the mobile computing device on or off. A microphone 1922 allows the mobile computing device 1910 to convert audible sounds into an electrical signal that may be digitally encoded and stored in computer-readable memory, or transmitted to another computing device. The mobile computing device 1910 may also include a digital compass, an accelerometer, proximity sensors, and ambient light sensors.

An operating system may provide an interface between the mobile computing device's hardware (e.g., the input/output mechanisms and a processor executing instructions retrieved from computer-readable medium) and software. Example operating systems include ANDROID, CHROME, IOS, MAC OS X, WINDOWS 7, WINDOWS PHONE 7, SYMBIAN, BLACKBERRY, WEBOS, a variety of UNIX operating systems; or a proprietary operating system for computerized devices. The operating system may provide a platform for the execution of application programs that facilitate interaction between the computing device and a user.

The mobile computing device 1910 may present a graphical user interface with the touchscreen 1912. A graphical user interface is a collection of one or more graphical interface elements and may be static (e.g., the display appears to remain the same over a period of time), or may be dynamic (e.g., the graphical user interface includes graphical interface elements that animate without user input).

A graphical interface element may be text, lines, shapes, images, or combinations thereof. For example, a graphical interface element may be an icon that is displayed on the desktop and the icon's associated text. In some examples, a graphical interface element is selectable with user-input. For example, a user may select a graphical interface element by pressing a region of the touchscreen that corresponds to a display of the graphical interface element. In some examples, the user may manipulate a trackball to highlight a single graphical interface element as having focus. User-selection of a graphical interface element may invoke a pre-defined action by the mobile computing device. In some examples, selectable graphical interface elements further or alternatively correspond to a button on the keyboard 1914. User-selection of the button may invoke the pre-defined action.

In some examples, the operating system provides a “desktop” graphical user interface that is displayed after turning on the mobile computing device 1910, after activating the mobile computing device 1910 from a sleep state, after “unlocking” the mobile computing device 1910, or after receiving user-selection of the “home” button 1918c. The desktop graphical user interface may display several graphical interface elements that, when selected, invoke corresponding application programs. An invoked application program may present a graphical interface that replaces the desktop graphical user interface until the application program terminates or is hidden from view.

User-input may influence an executing sequence of mobile computing device 1910 operations. For example, a single-action user input (e.g., a single tap of the touchscreen, swipe across the touchscreen, contact with a button, or combination of these occurring at a same time) may invoke an operation that changes a display of the user interface. Without the user-input, the user interface may not have changed at a particular time. For example, a multi-touch user input with the touchscreen 1912 may invoke a mapping application to “zoom-in” on a location, even though the mapping application may have by default zoomed-in after several seconds.

The desktop graphical interface can also display “widgets.” A widget is one or more graphical interface elements that are associated with an application program that is executing, and that display on the desktop content controlled by the executing application program. A widget's application program may launch as the mobile device turns on. Further, a widget may not take focus of the full display. Instead, a widget may only “own” a small portion of the desktop, displaying content and receiving touchscreen user-input within the portion of the desktop.

The mobile computing device 1910 may include one or more location-identification mechanisms. A location-identification mechanism may include a collection of hardware and software that provides the operating system and application programs an estimate of the mobile device's geographical position. A location-identification mechanism may employ satellite-based positioning techniques, base station transmitting antenna identification, multiple base station triangulation, internet access point IP location determinations, inferential identification of a user's position based on search engine queries, and user-supplied identification of location (e.g., by receiving user a “check in” to a location).

The mobile computing device 1910 may include other applications, computing sub-systems, and hardware. A call handling unit may receive an indication of an incoming telephone call and provide a user the capability to answer the incoming telephone call. A media player may allow a user to listen to music or play movies that are stored in local memory of the mobile computing device 1910. The mobile computing device 1910 may include a digital camera sensor, and corresponding image and video capture and editing software. An internet browser may enable the user to view content from a web page by typing in an addresses corresponding to the web page or selecting a link to the web page.

The mobile computing device 1910 may include an antenna to wirelessly communicate information with the base station 1940. The base station 1940 may be one of many base stations in a collection of base stations (e.g., a mobile telephone cellular network) that enables the mobile computing device 1910 to maintain communication with a network 1950 as the mobile computing device is geographically moved. The computing device 1910 may alternatively or additionally communicate with the network 1950 through a Wi-Fi router or a wired connection (e.g., ETHERNET, USB, or FIREWIRE). The computing device 1910 may also wirelessly communicate with other computing devices using BLUETOOTH protocols, or may employ an ad-hoc wireless network.

A service provider that operates the network of base stations may connect the mobile computing device 1910 to the network 1950 to enable communication between the mobile computing device 1910 and other computing systems that provide services 1960. Although the services 1960 may be provided over different networks (e.g., the service provider's internal network, the Public Switched Telephone Network, and the Internet), network 1950 is illustrated as a single network. The service provider may operate a server system 1952 that routes information packets and voice data between the mobile computing device 1910 and computing systems associated with the services 1960.

The network 1950 may connect the mobile computing device 1910 to the Public Switched Telephone Network (PSTN) 1962 in order to establish voice or fax communication between the mobile computing device 1910 and another computing device. For example, the service provider server system 1952 may receive an indication from the PSTN 1962 of an incoming call for the mobile computing device 1910. Conversely, the mobile computing device 1910 may send a communication to the service provider server system 1952 initiating a telephone call using a telephone number that is associated with a device accessible through the PSTN 1962.

The network 1950 may connect the mobile computing device 1910 with a Voice over Internet Protocol (VoIP) service 1964 that routes voice communications over an IP network, as opposed to the PSTN. For example, a user of the mobile computing device 1910 may invoke a VoIP application and initiate a call using the program. The service provider server system 1952 may forward voice data from the call to a VoIP service, which may route the call over the internet to a corresponding computing device, potentially using the PSTN for a final leg of the connection.

An application store 1966 may provide a user of the mobile computing device 1910 the ability to browse a list of remotely stored application programs that the user may download over the network 1950 and install on the mobile computing device 1910. The application store 1966 may serve as a repository of applications developed by third-party application developers. An application program that is installed on the mobile computing device 1910 may be able to communicate over the network 1950 with server systems that are designated for the application program. For example, a VoIP application program may be downloaded from the Application Store 1966, enabling the user to communicate with the VoIP service 1964.

The mobile computing device 1910 may access content on the internet 1968 through network 1950. For example, a user of the mobile computing device 1910 may invoke a web browser application that requests data from remote computing devices that are accessible at designated universal resource locations. In various examples, some of the services 1960 are accessible over the internet.

The mobile computing device may communicate with a personal computer 1970. For example, the personal computer 1970 may be the home computer for a user of the mobile computing device 1910. Thus, the user may be able to stream media from his personal computer 1970. The user may also view the file structure of his personal computer 1970, and transmit selected documents between the computerized devices.

A voice recognition service 1972 may receive voice communication data recorded with the mobile computing device's microphone 1922, and translate the voice communication into corresponding textual data. In some examples, the translated text is provided to a search engine as a web query, and responsive search engine search results are transmitted to the mobile computing device 1910.

The mobile computing device 1910 may communicate with a social network 1974. The social network may include numerous members, some of which have agreed to be related as acquaintances. Application programs on the mobile computing device 1910 may access the social network 1974 to retrieve information based on the acquaintances of the user of the mobile computing device. For example, an “address book” application program may retrieve telephone numbers for the user's acquaintances. In various examples, content may be delivered to the mobile computing device 1910 based on social network distances from the user to other members in a social network graph of members and connecting relationships. For example, advertisement and news article content may be selected for the user based on a level of interaction with such content by members that are “close” to the user (e.g., members that are “friends” or “friends of friends”).

The mobile computing device 1910 may access a personal set of contacts 1976 through network 1950. Each contact may identify an individual and include information about that individual (e.g., a phone number, an email address, and a birthday). Because the set of contacts is hosted remotely to the mobile computing device 1910, the user may access and maintain the contacts 1976 across several devices as a common set of contacts.

The mobile computing device 1910 may access cloud-based application programs 1978. Cloud-computing provides application programs (e.g., a word processor or an email program) that are hosted remotely from the mobile computing device 1910, and may be accessed by the device 1910 using a web browser or a dedicated program. Example cloud-based application programs include GOOGLE DOCS word processor and spreadsheet service, GOOGLE GMAIL webmail service, and PICASA picture manager.

Mapping service 1980 can provide the mobile computing device 1910 with street maps, route planning information, and satellite images. An example mapping service is GOOGLE MAPS. The mapping service 1980 may also receive queries and return location-specific results. For example, the mobile computing device 1910 may send an estimated location of the mobile computing device and a user-entered query for “pizza places” to the mapping service 1980. The mapping service 1980 may return a street map with “markers” superimposed on the map that identify geographical locations of nearby “pizza places.”

Turn-by-turn service 1982 may provide the mobile computing device 1910 with turn-by-turn directions to a user-supplied destination. For example, the turn-by-turn service 1982 may stream to device 1910 a street-level view of an estimated location of the device, along with data for providing audio commands and superimposing arrows that direct a user of the device 1910 to the destination.

Various forms of streaming media 1984 may be requested by the mobile computing device 1910. For example, computing device 1910 may request a stream for a pre-recorded video file, a live television program, or a live radio program. Example services that provide streaming media include YOUTUBE and PANDORA.

A micro-blogging service 1986 may receive from the mobile computing device 1910 a user-input post that does not identify recipients of the post. The micro-blogging service 1986 may disseminate the post to other members of the micro-blogging service 1986 that agreed to subscribe to the user.

A search engine 1988 may receive user-entered textual or verbal queries from the mobile computing device 1910, determine a set of internet-accessible documents that are responsive to the query, and provide to the device 1910 information to display a list of search results for the responsive documents. In examples where a verbal query is received, the voice recognition service 1972 may translate the received audio into a textual query that is sent to the search engine.

These and other services may be implemented in a server system 1990. A server system may be a combination of hardware and software that provides a service or a set of services. For example, a set of physically separate and networked computerized devices may operate together as a logical server system unit to handle the operations necessary to offer a service to hundreds of computing devices. A server system is also referred to herein as a computing system.

In various implementations, operations that are performed “in response to” or “as a consequence of” another operation (e.g., a determination or an identification) are not performed if the prior operation is unsuccessful (e.g., if the determination was not performed). Operations that are performed “automatically” are operations that are performed without user intervention (e.g., intervening user input). Features in this document that are described with conditional language may describe implementations that are optional. In some examples, “transmitting” from a first device to a second device includes the first device placing data into a network for receipt by the second device, but may not include the second device receiving the data. Conversely, “receiving” from a first device may include receiving the data from a network, but may not include the first device transmitting the data.

“Determining” by a computing system can include the computing system requesting that another device perform the determination and supply the results to the computing system. Moreover, “displaying” or “presenting” by a computing system can include the computing system sending data for causing another device to display or present the referenced information.

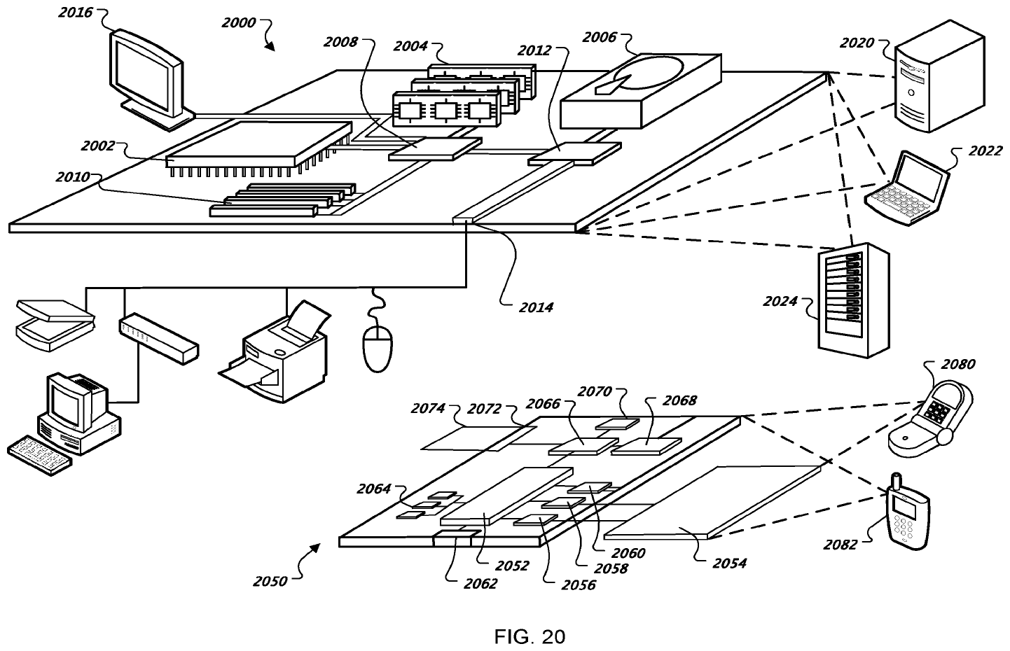

FIG. 20 is a block diagram of computing devices 2000, 2050 that may be used to implement the systems and methods described in this document, as either a client or as a server or plurality of servers. Computing device 2000 is intended to represent various forms of digital computers, such as laptops, desktops, workstations, personal digital assistants, servers, blade servers, mainframes, and other appropriate computers. Computing device 2050 is intended to represent various forms of mobile devices, such as personal digital assistants, cellular telephones, smartphones, and other similar computing devices. The components shown here, their connections and relationships, and their functions, are meant to be examples only, and are not meant to limit implementations described and/or claimed in this document.

Computing device 2000 includes a processor 2002, memory 2004, a storage device 2006, a high-speed controller 2008 connecting to memory 2004 and high-speed expansion ports 2010, and a low speed controller 2012 connecting to low speed expansion port 2014 and storage device 2006. Each of the components 2002, 2004, 2006, 2008, 2010, and 2012, are interconnected using various busses, and may be mounted on a common motherboard or in other manners as appropriate. The processor 2002 can process instructions for execution within the computing device 2000, including instructions stored in the memory 2004 or on the storage device 2006 to display graphical information for a GUI on an external input/output device, such as display 2016 coupled to high-speed controller 2008. In other implementations, multiple processors and/or multiple buses may be used, as appropriate, along with multiple memories and types of memory. Also, multiple computing devices 2000 may be connected, with each device providing portions of the necessary operations (e.g., as a server bank, a group of blade servers, or a multi-processor system).

The memory 2004 stores information within the computing device 2000. In one implementation, the memory 2004 is a volatile memory unit or units. In another implementation, the memory 2004 is a non-volatile memory unit or units. The memory 2004 may also be another form of computer-readable medium, such as a magnetic or optical disk.

The storage device 2006 is capable of providing mass storage for the computing device 2000. In one implementation, the storage device 2006 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 2004, the storage device 2006, or memory on processor 2002.

The high-speed controller 2008 manages bandwidth-intensive operations for the computing device 2000, while the low speed controller 2012 manages lower bandwidth-intensive operations. Such allocation of functions is an example only. In one implementation, the high-speed controller 2008 is coupled to memory 2004, display 2016 (e.g., through a graphics processor or accelerator), and to high-speed expansion ports 2010, which may accept various expansion cards (not shown). In the implementation, low-speed controller 2012 is coupled to storage device 2006 and low-speed expansion port 2014. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 2000 may be implemented in a number of different forms, as shown in the figure. For example, it may be implemented as a standard server 2020, or multiple times in a group of such servers. It may also be implemented as part of a rack server system 2024. In addition, it may be implemented in a personal computer such as a laptop computer 2022. Alternatively, components from computing device 2000 may be combined with other components in a mobile device (not shown), such as device 2050. Each of such devices may contain one or more of computing device 2000, 2050, and an entire system may be made up of multiple computing devices 2000, 2050 communicating with each other.

Computing device 2050 includes a processor 2052, memory 2064, an input/output device such as a display 2054, a communication interface 2066, and a transceiver 2068, among other components. The device 2050 may also be provided with a storage device, such as a microdrive or other device, to provide additional storage. Each of the components 2050, 2052, 2064, 2054, 2066, and 2068, are interconnected using various buses, and several of the components may be mounted on a common motherboard or in other manners as appropriate.

The processor 2052 can execute instructions within the computing device 2050, including instructions stored in the memory 2064. The processor may be implemented as a chipset of chips that include separate and multiple analog and digital processors. Additionally, the processor may be implemented using any of a number of architectures. For example, the processor may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 2050, such as control of user interfaces, applications run by device 2050, and wireless communication by device 2050.

Processor 2052 may communicate with a user through control interface 2058 and display interface 2056 coupled to a display 2054. The display 2054 may be, for example, a TFT (Thin-Film-Transistor Liquid Crystal Display) display or an OLED (Organic Light Emitting Diode) display, or other appropriate display technology. The display interface 2056 may comprise appropriate circuitry for driving the display 2054 to present graphical and other information to a user. The control interface 2058 may receive commands from a user and convert them for submission to the processor 2052. In addition, an external interface 2062 may be provide in communication with processor 2052, so as to enable near area communication of device 2050 with other devices. External interface 2062 may provided, for example, for wired communication in some implementations, or for wireless communication in other implementations, and multiple interfaces may also be used.

The memory 2064 stores information within the computing device 2050. The memory 2064 can be implemented as one or more of a computer-readable medium or media, a volatile memory unit or units, or a non-volatile memory unit or units. Expansion memory 2074 may also be provided and connected to device 2050 through expansion interface 2072, which may include, for example, a SIMM (Single In Line Memory Module) card interface. Such expansion memory 2074 may provide extra storage space for device 2050, or may also store applications or other information for device 2050. Specifically, expansion memory 2074 may include instructions to carry out or supplement the processes described above, and may include secure information also. Thus, for example, expansion memory 2074 may be provide as a security module for device 2050, and may be programmed with instructions that permit secure use of device 2050. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 2064, expansion memory 2074, or memory on processor 2052 that may be received, for example, over transceiver 2068 or external interface 2062.

Device 2050 may communicate wirelessly through communication interface 2066, which may include digital signal processing circuitry where necessary. Communication interface 2066 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 2068. In addition, short-range communication may occur, such as using a Bluetooth, WiFi, or other such transceiver (not shown). In addition, GPS (Global Positioning System) receiver module 2070 may provide additional navigation- and location-related wireless data to device 2050, which may be used as appropriate by applications running on device 2050.

Device 2050 may also communicate audibly using audio codec 2060, which may receive spoken information from a user and convert it to usable digital information. Audio codec 2060 may likewise generate audible sound for a user, such as through a speaker, e.g., in a handset of device 2050. Such sound may include sound from voice telephone calls, may include recorded sound (e.g., voice messages, music files, etc.) and may also include sound generated by applications operating on device 2050.

The computing device 2050 may be implemented in a number of different forms, as shown in the figure. For example, it may be implemented as a cellular telephone 2080. It may also be implemented as part of a smartphone 2082, personal digital assistant, or other similar mobile device.

Additionally computing device 2000 or 2050 can include Universal Serial Bus (USB) flash drives. The USB flash drives may store operating systems and other applications. The USB flash drives can include input/output components, such as a wireless transmitter or USB connector that may be inserted into a USB port of another computing device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

As additional description to the embodiments described above, the present disclosure describes the following embodiments.

### First Set of Embodiments—Chat-Style Interactive Q&A Session

The Assessment user interface screens of FIGS. 5A-F (and the descriptions thereof) provide additional detail regarding the embodiments listed below.

Embodiment 1 is a computer-implemented method of operations performed as part of a computer-human interactive session, the method comprising: presenting, by a computing device, a first user prompt on a touchscreen display device; presenting, by the computing device, at least two of multiple candidate user responses to the first user prompt below the first user prompt on the touchscreen display device; receiving, by the computing device, first user input that selects a first selected user response of the multiple candidate user responses to the first user prompt; presenting, by the computing device, the first selected user response beneath the first user prompt on the touchscreen display device, with other of the multiple candidate user responses to the first user prompt having been removed from the touchscreen display device responsive to receiving the first user input that selects the first selected user response; presenting, by the computing device, a second user prompt on the touchscreen display device, beneath the first selected user response that is beneath the first user prompt; presenting, by the computing device, at least two of multiple candidate user responses to the second user prompt below the second user prompt on the touchscreen display device; receiving, by the computing device, second user input that selects a second selected user response of the multiple candidate user responses to the second user prompt; and presenting, by the computing device, the second selected user response beneath the second user prompt on the touchscreen display device, with other of the multiple candidate user responses to the second user prompt having been removed from the touchscreen display device responsive to receiving the second user input that selects the second selected user response.

Embodiment 2 is the computer-implemented method of embodiment 1, wherein: the multiple candidate user responses to the first user prompt are same as the multiple candidate user responses to the second user prompt; and the first selected user response is different from the second selected user response.

Embodiment 3 is the computer-implemented method of any of embodiments 1-2, wherein: receiving the first user input that selects the first selected user response includes receiving an indication that user input contacted a portion of the touchscreen display device at which the first selected user response was presented; and receiving the second user input that selects the second selected user response includes receiving an indication that user input contacted a portion of the touchscreen display device at which the second selected user response was presented.

Embodiment 4 is the computer-implemented method of any of embodiments 1-3, wherein: the first user prompt comprises text located within a first interface element that has its center offset left of center of the touchscreen display device; the first selected user response comprises text located within a second interface element that has its center offset right of center of the touchscreen display device; the second user prompt comprises text located within a third interface element that has its center offset left of center of the touchscreen display device; and the second selected user response comprises text located within a fourth interface element that has its center offset right of center of the touchscreen display device.

Embodiment 5 is the computer-implemented method of any of embodiments 1-4, wherein: the at least two of multiple candidate user responses to the first user prompt that are presented below the first user prompt represent a first subset of the multiple candidate user responses to the first user prompt; the method comprises receiving, by the computing device, user input that moves across a portion of the touchscreen display at which the first subset of the multiple candidate user responses are displayed, and in response, replacing the presentation of the first subset of the multiple candidate user responses with a different, second subset of the multiple candidate user responses.

Embodiment 6 is the computer-implemented method of any of embodiments 1-5, wherein: the computing device presents the first selected user response with its center at a first horizontal offset from a center of the touchscreen prior to the first user input selecting the first selected user response; and the computing device presents the first selected user response with its center at a second horizontal offset from the center of the touchscreen after the first user input has selected the first selected user response, the second horizontal offset being different from the first horizontal offset.

Embodiment 7 is the computer-implemented method of any of embodiments 1-6, wherein: the computing device does not present the second user prompt on the touchscreen display device until after receipt of the first user input that selects the first selected user response.

Embodiment 8 is the computer-implemented method of any of embodiments 1-7, wherein: all other of the multiple candidate user responses to the first user prompt are removed from the touchscreen display device responsive to receiving the first user input that selects the first selected user response; and all other of the multiple candidate user responses to the second user prompt are removed from the touchscreen display device responsive to receiving the second user input that selects the second selected user response.

Embodiment 9 is the computer-implemented method of any of embodiments 1-8, comprising: determining, by the computing device, a single value for a metric that describes a characteristic of a user of the computing device based on both the first selected user response and the second selected user response.

Embodiment 10 is the computer-implemented method of any of embodiments 1-9, comprising: receiving, by the computing device, user input that re-selects the first selected user response, after the other of the multiple candidate user responses to the first user prompt have been removed from the touchscreen display device, and in response re-presenting the at least two of the multiple candidate user responses to the first user prompt; receiving, by the computing device after receiving the user input that re-selects the first user response, user input that selects a different first selected user response to the first prompt; and presenting, by the computing device, the different first selected user response beneath the first user prompt on the touchscreen display device, with other of the multiple candidate user responses to the first user prompt having been removed from the touchscreen display device, responsive to receiving the first user input that re-selects the first selected user response.

Embodiment 11 is the computer-implemented method of any of embodiments 1-10, comprising: presenting, by the computing device, a third user prompt on the touchscreen display device, beneath the second selected user response that is beneath the second user prompt that is beneath the first selected user response that is beneath the first user prompt; presenting, by the computing device, at least two of multiple candidate user responses to the third user prompt below the third user prompt on the touchscreen display device; receiving, by the computing device, third user input that selects a third selected user response of the multiple candidate user responses to the third user prompt; and presenting, by the computing device, the third selected user response beneath the third user prompt on the touchscreen display device, with other of the multiple candidate user responses to the third user prompt having been removed from the touchscreen display device, responsive to receiving the third user input that selects the third selected user response.

Embodiment 12 is the computing system, comprising: one or more processors; and one or more computer-readable devices including instructions stored thereon that, when executed by the one or more processors, cause the computing system to perform the method of any of embodiments 1-11.

### Second Set of Embodiments—User Interaction Changes Both User Response and Graphical Element

The user interface screens of FIGS. 8A-D (waves), 10A-L (human head), 14A-M (tree), and 16C-O (dots) (and the descriptions thereof) provide additional detail regarding the embodiments listed below.

Embodiment 1 is a computer-implemented method of operations performed as part of a computer-human interactive session, the method comprising: presenting, by a computing device, a first statement on a touchscreen display device; presenting, by the computing device, a first user response that includes first text indicating a first extent of user agreeableness with the first statement; presenting, by the computing device, one or more first graphical interface elements having a first configuration that graphically indicates the first extent of user agreeableness with the first statement; receiving, by the computing device, an indication that first user input contacts the touchscreen display device and moves across the touchscreen display device from a first location to a second location while remaining in contact with the touchscreen display device; changing, by the computing device, the presentation of the first user response from including the first text to including different, second text indicating a different, second extent of user agreeableness with the first statement, responsive to the first user input having moved from the first location to the second location; changing, by the computing device, the presentation of the one or more first graphical interface elements from having the first configuration to having a second configuration that graphically indicates the second extent of user agreeableness with the first statement, responsive to the first user input having moved from the first location to the second location; receiving, by the computing device, an indication that the first user input has moved across the touchscreen display device from the second location to a third location while remaining in contact with the touchscreen display device; changing, by the computing device, the presentation of the first user response from including the second text to including different, third text indicating a different, third extent of user agreeableness with the first statement, responsive to the first user input having moved from the second location to the third location; and changing, by the computing device, the presentation of the one or more first graphical interface elements from having the second configuration to having a third configuration that graphically indicates the third extent of user agreeableness with the first statement, responsive to the first user input having moved from the second location to the third location.

Embodiment 2 is the computer-implemented method of embodiment 1, wherein: the computing device presents the first text concurrent with the one or more first graphical interface elements having the first configuration; the computing device presents the second text concurrent with the one or more first graphical interface elements having the second configuration; and the computing device presents the third text concurrent with the one or more first graphical interface elements having the third configuration.

Embodiment 3 is the computer-implemented method of any of embodiments 1-2, wherein: the first location, the second location, and the third location are located along a continuum, such that the second location is located between the first location and the third location; and the first text, the second text, and the third text indicate a continuum of user agreeableness with the first statement, such that the first text indicates less agreeableness with the first statement than the second text, and the third text indicates more agreeableness with the first statement than the second text.

Embodiment 4 is the computer-implemented method of any of embodiments 1-3, wherein: receiving the indication that first user input contacts the touchscreen display device and moves across the touchscreen display device from the first location to the second location includes the first user input contacting a control element and moving a displayed position of the control element from the first location to the second location; and the control element is distinct from the one or more graphical elements.

Embodiment 5 is the computer-implemented method of embodiment 4, wherein: the one or more first graphical elements do not move in a same direction and amount as the control element, responsive to the first user input moving the displayed position of the control element from the first location to the second location.

Embodiment 6 is the computer-implemented method of embodiment 4, wherein: the control element is presented along a displayed line that represents a path across which the first user input is able to move the control element.

Embodiment 7 is the computer-implemented method of embodiment 4, wherein: the first user input moves across at least a portion of the one or more first graphical elements, such that the control element is presented as superimposed over at least the portion of the one or more first graphical elements.

Embodiment 8 is the computer-implemented method of any of embodiments 1-7, comprising: receiving, by the computing device an indication that the first user input has released from the touchscreen display device at the third location; receiving, by the computing device, user selection of an interface element to transition the touchscreen from presenting a first interface screen that includes the first statement to presenting a second interface screen; and assigning, by the computing device, the third extent of user agreeableness with the first statement as the first user response to the first statement.

Embodiment 9 is the computer-implemented method of embodiment 8, comprising: presenting, by the computing device, a second statement on the second interface screen; presenting, by the computing device, a second user response that includes same said first text indicating the first extent of user agreeableness with the first statement; presenting, by the computing device, the one or more first graphical interface elements in the first configuration that graphically indicates the first extent of user agreeableness with the second statement; receiving, by the computing device, an indication that second user input contacts the touchscreen display device and moves across the touchscreen display device from a fourth location to a fifth location while remaining in contact with the touchscreen display device; changing, by the computing device, the presentation of the second user response from including the first text to including the second text indicating the second extent of user agreeableness with the first statement, responsive to the second user input having moved from the fourth location to the fifth location; and changing, by the computing device, the presentation of the one or more first graphical interface elements from having the first configuration to having the second configuration that graphically indicates the second extent of user agreeableness with the second statement, responsive to the second user input having moved from the fourth location to the fifth location.

Embodiment 10 is the computer-implemented method of any of embodiments 1-9, wherein: the one or more first graphical interface elements comprise one or more lines; the one or more first graphical interface elements having the first configuration that graphically indicates the first extent of user agreeableness with the first statement includes the set of one or more lines having a first level of waviness; the one or more first graphical interface elements having the second configuration that graphically indicates the second extent of user agreeableness with the first statement includes the set of one or more lines having a second level of waviness with greater waviness than the first level of waviness; and the one or more first graphical interface elements having the third configuration that graphically indicates the third extent of user agreeableness with the first statement includes the set of one or more lines having a third level of waviness with greater waviness than the second level of waviness.

Embodiment 11 is the computer-implemented method of any of embodiments 1-9, wherein: the one or more first graphical interface elements graphically depict an object; the one or more first graphical interface elements having the first configuration that graphically indicates the first extent of user agreeableness with the first statement includes the object being depicted with a first level of detail; the one or more first graphical interface elements having the second configuration that graphically indicates the second extent of user agreeableness with the first statement includes the object being depicted with a second level of detail that is greater than the first level of detail; and the one or more first graphical interface elements having the third configuration that graphically indicates the third extent of user agreeableness with the first statement includes the object being depicted with a third level of detail that is greater than the second level of detail.

Embodiment 12 is a computing system, comprising: one or more processors; and one or more computer-readable devices including instructions stored thereon that, when executed by the one or more processors, cause the computing system to perform the method of any of embodiments 1-11.

### Third Set of Embodiments—Determination of Sleep Metric

The user interface screens of FIGS. 3, 4A-F, and 6A-C (and the

descriptions thereof), and the Biometrics section in the above disclosure, provide additional detail regarding the embodiments listed below.

Embodiment 1 is a computer-implemented method for determining quality of user sleep, comprising: receiving, by a computing system, an indication that user input interacted with a computing device to indicate that a user is starting a process for going to sleep; recording, by the computing system, a start time that corresponds to a time at which the user input interacted with the computing device to indicate that the user is starting the process for going to sleep; identifying, by the computing system, an end time at which the user awoke from sleep, the end time having been determined based on measurements from one or more biometric sensors configured to measure physical characteristics of the user; and determining, by the computing system, a level of sleep quality for the sleep of the user based on the start time and the end time.

Embodiment 2 is the computer-implemented method of embodiment 1, wherein: the user input that indicated that the user is starting the process for going to sleep includes user selection of an interface element that is: presented by an application program configured to present an indication of the level of sleep quality determined by the computing system based on the start time and the end time, and designated by the application program for user selection upon the user starting the process for going to sleep. Embodiment 3 is the computer-implemented method of any of embodiments 1-2, comprising: identifying, by the computing system, a first fallen asleep time at which the user first fell asleep after having provided the user input that indicated that the user is starting the process for going to sleep, the first fallen asleep time having been determined based on measurements from one or more biometric sensors configured to measure physical characteristics of the user; and determining, by the computing system, a length of time that it took the user to fall asleep, based on a comparison of the start time to the first fallen asleep time, wherein determining the level of sleep quality is based on the determined length of time that it took the user to fall asleep.

Embodiment 4 is the computer-implemented method of any of embodiments 1-3, wherein the end time was determined based on: measurements from an accelerometer in a user-wearable computing device exceeding a threshold level of activity for a threshold duration of time; and measurements from a gyroscope in the user-wearable computing device indicating that the user has transitioned from laying down to being oriented upright.

Embodiment 5 is the computer-implemented method of embodiment 4, wherein the user-wearable computing device comprises a watch worn by the user during the sleep.

Embodiment 6 is the computer-implemented method of any of embodiments 1-5, comprising: identifying, by the computing system, multiple awaken times at which the user awoke from sleep at different times during sleep, with each awaken time of the multiple awaken times having been determined based on measurements from the one or more biometric sensors indicating that device movement exceeded a threshold level of movement; and identifying, by the computing system, multiple fallen-back-asleep times at which the user fell back to sleep at different times after having awoken from sleep, with each fallen-back-asleep time having been determined based on measurements from the one or more biometric sensors indicating that device movement fell below a certain level of movement, wherein determining the level of sleep quality is based on analysis of the identified multiple awaken times and the identified multiple fallen-back-asleep times.

Embodiment 7 is the computer-implemented method of embodiment 6, comprising: determining, by the computing system, a quantity of times that the user awoke and fell back asleep during the sleep, based on analysis of the multiple awaken times and the multiple fallen-back-asleep times, wherein determining the level of sleep quality is based on the determined quantity of times that the user awoke and fell back asleep.

Embodiment 8 is the computer-implemented method of embodiment 6, comprising: identifying, by the computing system, a first fallen asleep time at which the user first fell asleep after having provided the user input that indicated that the user is starting the process for going to sleep, the first fallen asleep time having been determined based on measurements from the one or more biometric sensors configured to measure physical characteristics of the user; and determining, by the computing system, a proportion of time between the start time and the end time that the user was sleeping, based on analysis of: (i) the start time that corresponds to the time at which the user input interacted with the computing device to indicate that the user is starting the process for going to sleep; (ii) the identified first fallen asleep time at which the user first fell asleep: (iii) the identified multiple awaken times; (iv) the identified multiple fallen-back-asleep times; and (v) the end time at which the user awoke from sleep, wherein determining the level of sleep quality is based on the determined proportion of time between the start time and the end time that the user was sleeping.

Embodiment 9 is the computer-implemented method of any of embodiments 1-8, comprising: determining, by the computing system, a pre-sleep time period extending from: (i) a certain amount of time prior to the start time that corresponds to the time at which the user input interacted with the computing device to indicate that the user is starting the process for going to sleep; and (ii) the start time; determining, by the computing system, a pre-sleep heart rate based on measurements obtained from a heart-rate sensor over the pre-sleep time period; determining, by the computing system, a while-sleeping heart rate based on measurements obtained from the heart-rate sensor between the start time and the end time; and determining, by the computing system, an extent to which the while-sleeping heart rate drops below the pre-sleep heart rate, wherein determining the level of sleep quality is based on the determined extent to which the while-sleeping heart rate drops below the pre-sleep heart rate.

Embodiment 10 is the computer-implemented method of any of embodiments 1-9, comprising: identifying, by the computing system, that measurements from the one or more biometric sensors indicate that the user has risen from bed a first time; presenting, by the computing system, a first prompt that the user specify whether the user has woken or whether the user is going to continue with the sleep; receiving, by the computing system responsive to presentation of the first prompt, first user input selecting an interface element to indicate that the user is going to continue with the sleep; identifying, by the computing system, that measurements from the one or more biometric sensors indicate that the user has risen from bed a second time that follows the first time; presenting, by the computing system, a second prompt that the user specify whether the user has woken or whether the user is going to continue with the sleep; receiving, by the computing system responsive to presentation of the second prompt, second user input selecting an interface element to indicate that the user has woken; and designating, by the computing system, the end time as a time at which the user rose from bed for the second time, responsive to the second user input selecting the interface element to indicate that the user has woken.

Embodiment 11 is the computer-implemented method of any one of embodiments 1-10, comprising: presenting, by the computing system, a graphical depiction of the level of sleep quality for the sleep and graphical depictions of a level of sleep quality for each of multiple previous sleeps, to graphically illustrate historical trends in sleep quality of the user.

Embodiment 12 is a computing system, comprising: one or more processors; and one or more computer-readable devices including instructions stored thereon that, when executed by the one or more processors, cause the computing system to perform the method of any of embodiments 1-11.

### Fourth Set of Embodiments—Odyssey Human/Computer Interactive Experience

The user interface screens of FIGS. 12A-E, 13A-E, 14A-M, and 15A-C (and the descriptions thereof) provide additional detail regarding the embodiments listed below.

Embodiment 1 is a computer-implemented method, comprising: presenting, by a computing system on a touchscreen display device, for each respective event of multiple different events that are potentially relevant to a user of the computing system: (i) text that identifies a type of the respective event; and (ii) an event-relevant selectable interface element; receiving, by the computing system, user input that selects a subset of selected events of the multiple different events, by user selection of the event-relevant selectable interface element that corresponds to each selected event in the subset of selected events; presenting, by the computing system for each selected event in the subset of selected events: (i) an intensity-identifying interface element that is user selectable to select one of multiple different levels of intensity for the respective selected event; and (ii) one or more type-of-effect interface elements that are user selectable to indicate whether an effect of the selected event on the user is negative or positive; receiving, by the computing system for each selected event in the subset of selected events: (i) user input that interacts with the intensity-identifying interface element to select a selected level of intensity for the respective selected event; and (ii) user input that interacts with the one or more type-of-effect interface elements to select a selected type of effect of the selected event on the user as being negative or positive; analyzing, by the computing system, the selected level of intensity and the selected type of effect for each selected event in the subset of selected events; and presenting, by the computing system, an interface that indicates an impact of each selected event in the subset of selected events on the user, based on the selected level of intensity and the selected type of effect for each selected event.

Embodiment 2 is the computer-implemented method of embodiment 1, wherein: the multiple different levels of intensity that are user selectable for each selected event includes at least five different levels of intensity.

Embodiment 3 is the computer-implemented method of any of embodiments 1-2, wherein presenting the interface that indicates the impact of each selected event on the user includes presenting a graph that indicates how the selected level of intensity for a particular selected event in the subset of selected events changes over time, based on multiple different user selections of the selected level of intensity for the particular selected event at different times.

Embodiment 4 is the computer-implemented method of any of embodiments 1-3, comprising: presenting, by the computing system after the computing system has identified the selected level of intensity and the selected type of effect for each selected event in the subset of selected events, a user interface that includes, for each selected event in the subset of selected events, a begin-assessment interface element; receiving, by the computing system, user input that selects the begin-assessment interface element for a certain selected event from the subset of selected events; presenting, by the computing system, a series of multiple statements directed to an effect of the certain selected event on the user, with each statement in the series of multiple statements accompanied by a user interface element that enables user input to select an extent to which the user agrees with the statement; and receiving, by the computing system, user inputs that interact with the user interface elements that accompany the multiple statements to specify the extent to which the user agrees with each statement of the multiple statements.

Embodiment 5 is the computer-implemented method of embodiment 4, wherein the series of multiple statements includes at least five statements.

Embodiment 6 is the computer-implemented method of embodiment 4, comprising: analyzing, by the computing system, the user inputs that interact with the interface elements that accompany the multiple statements to determine one category of multiple categories into which behavior of the user is categorized; and presenting, by the computing system on the touchscreen display device, stored information that describes aspects of the one category into which behavior of the user is categorized.

Embodiment 7 is a computing system, comprising: one or more processors; and one or more computer-readable devices including instructions stored thereon that, when executed by the one or more processors, cause the computing system to perform the method of any of embodiments 1-6.

Although a few implementations have been described in detail above, other modifications are possible. Moreover, other mechanisms for performing the systems and methods described in this document may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following embodiments.

1-12. (canceled)

13. A computer-implemented method of operations performed as part of a computer-human interactive session, the method comprising:
presenting, by a computing device, a first statement on a touchscreen display device;
presenting, by the computing device, a first user response that includes first text indicating a first extent of user agreeableness with the first statement;
presenting, by the computing device, one or more first graphical interface elements having a first configuration that graphically indicates the first extent of user agreeableness with the first statement;
receiving, by the computing device, an indication that first user input contacts the touchscreen display device and moves across the touchscreen display device from a first location to a second location while remaining in contact with the touchscreen display device;
changing, by the computing device, the presentation of the first user response from including the first text to including different, second text indicating a different, second extent of user agreeableness with the first statement, responsive to the first user input having moved from the first location to the second location;
changing, by the computing device, the presentation of the one or more first graphical interface elements from having the first configuration to having a second configuration that graphically indicates the second extent of user agreeableness with the first statement, responsive to the first user input having moved from the first location to the second location;
receiving, by the computing device, an indication that the first user input has moved across the touchscreen display device from the second location to a third location while remaining in contact with the touchscreen display device;
changing, by the computing device, the presentation of the first user response from including the second text to including different, third text indicating a different, third extent of user agreeableness with the first statement, responsive to the first user input having moved from the second location to the third location; and
changing, by the computing device, the presentation of the one or more first graphical interface elements from having the second configuration to having a third configuration that graphically indicates the third extent of user agreeableness with the first statement, responsive to the first user input having moved from the second location to the third location.

14. The computer-implemented method of claim 13, wherein:
the computing device presents the first text concurrent with the one or more first graphical interface elements having the first configuration;
the computing device presents the second text concurrent with the one or more first graphical interface elements having the second configuration; and
the computing device presents the third text concurrent with the one or more first graphical interface elements having the third configuration.

15. The computer-implemented method of claim 13, wherein:
the first location, the second location, and the third location are located along a continuum, such that the second location is located between the first location and the third location; and
the first text, the second text, and the third text indicate a continuum of user agreeableness with the first statement, such that the first text indicates less agreeableness with the first statement than the second text, and the third text indicates more agreeableness with the first statement than the second text.

16. The computer-implemented method of claim 13, wherein:
receiving the indication that first user input contacts the touchscreen display device and moves across the touchscreen display device from the first location to the second location includes the first user input contacting a control element and moving a displayed position of the control element from the first location to the second location; and
the control element is distinct from the one or more graphical elements.

17. The computer-implemented method of claim 16, wherein:
the one or more first graphical elements do not move in a same direction and amount as the control element, responsive to the first user input moving the displayed position of the control element from the first location to the second location.

18. The computer-implemented method of claim 16, wherein:
the control element is presented along a displayed line that represents a path across which the first user input is able to move the control element.

19. The computer-implemented method of claim 16, wherein:
the first user input moves across at least a portion of the one or more first graphical elements, such that the control element is presented as superimposed over at least the portion of the one or more first graphical elements.

20. The computer-implemented method of claim 13, comprising:
receiving, by the computing device an indication that the first user input has released from the touchscreen display device at the third location;
receiving, by the computing device, user selection of an interface element to transition the touchscreen from presenting a first interface screen that includes the first statement to presenting a second interface screen; and
assigning, by the computing device, the third extent of user agreeableness with the first statement as the first user response to the first statement.

21. The computer-implemented method of claim 20, comprising:
presenting, by the computing device, a second statement on the second interface screen;
presenting, by the computing device, a second user response that includes same said first text indicating the first extent of user agreeableness with the first statement;
presenting, by the computing device, the one or more first graphical interface elements in the first configuration that graphically indicates the first extent of user agreeableness with the second statement;
receiving, by the computing device, an indication that second user input contacts the touchscreen display device and moves across the touchscreen display device from a fourth location to a fifth location while remaining in contact with the touchscreen display device;
changing, by the computing device, the presentation of the second user response from including the first text to including the second text indicating the second extent of user agreeableness with the first statement, responsive to the second user input having moved from the fourth location to the fifth location; and
changing, by the computing device, the presentation of the one or more first graphical interface elements from having the first configuration to having the second configuration that graphically indicates the second extent of user agreeableness with the second statement, responsive to the second user input having moved from the fourth location to the fifth location.

22. The computer-implemented method of claim 13, wherein:
the one or more first graphical interface elements comprise one or more lines;
the one or more first graphical interface elements having the first configuration that graphically indicates the first extent of user agreeableness with the first statement includes the set of one or more lines having a first level of waviness;
the one or more first graphical interface elements having the second configuration that graphically indicates the second extent of user agreeableness with the first statement includes the set of one or more lines having a second level of waviness with greater waviness than the first level of waviness; and
the one or more first graphical interface elements having the third configuration that graphically indicates the third extent of user agreeableness with the first statement includes the set of one or more lines having a third level of waviness with greater waviness than the second level of waviness.

23. The computer-implemented method of claim 13, wherein:
the one or more first graphical interface elements graphically depict an object;
the one or more first graphical interface elements having the first configuration that graphically indicates the first extent of user agreeableness with the first statement includes the object being depicted with a first level of detail;
the one or more first graphical interface elements having the second configuration that graphically indicates the second extent of user agreeableness with the first statement includes the object being depicted with a second level of detail that is greater than the first level of detail; and
the one or more first graphical interface elements having the third configuration that graphically indicates the third extent of user agreeableness with the first statement includes the object being depicted with a third level of detail that is greater than the second level of detail.

24. A computing system, comprising:
one or more processors; and
one or more computer-readable devices including instructions stored thereon that, when executed by the one or more processors, cause the computing system to perform operations that comprise:
presenting, by the computing system, a first statement on a touchscreen display device;
presenting, by the computing system, a first user response that includes first text indicating a first extent of user agreeableness with the first statement;
presenting, by the computing system, one or more first graphical interface elements having a first configuration that graphically indicates the first extent of user agreeableness with the first statement;
receiving, by the computing system, an indication that first user input contacts the touchscreen display device and moves across the touchscreen display device from a first location to a second location while remaining in contact with the touchscreen display device;
changing, by the computing system, the presentation of the first user response from including the first text to including different, second text indicating a different, second extent of user agreeableness with the first statement, responsive to the first user input having moved from the first location to the second location;
changing, by the computing system, the presentation of the one or more first graphical interface elements from having the first configuration to having a second configuration that graphically indicates the second extent of user agreeableness with the first statement, responsive to the first user input having moved from the first location to the second location;
receiving, by the computing system, an indication that the first user input has moved across the touchscreen display device from the second location to a third location while remaining in contact with the touchscreen display device;
changing, by the computing system, the presentation of the first user response from including the second text to including different, third text indicating a different, third extent of user agreeableness with the first statement, responsive to the first user input having moved from the second location to the third location; and
changing, by the computing system, the presentation of the one or more first graphical interface elements from having the second configuration to having a third configuration that graphically indicates the third extent of user agreeableness with the first statement, responsive to the first user input having moved from the second location to the third location.

25-43. (canceled)

44. The computing system of claim 24, wherein:
the computing system presents the first text concurrent with the one or more first graphical interface elements having the first configuration;
the computing system presents the second text concurrent with the one or more first graphical interface elements having the second configuration; and
the computing system presents the third text concurrent with the one or more first graphical interface elements having the third configuration.

45. The computing system of claim 24, wherein:
the first location, the second location, and the third location are located along a continuum, such that the second location is located between the first location and the third location; and
the first text, the second text, and the third text indicate a continuum of user agreeableness with the first statement, such that the first text indicates less agreeableness with the first statement than the second text, and the third text indicates more agreeableness with the first statement than the second text.

46. The computing system of claim 24, wherein:
receiving the indication that first user input contacts the touchscreen display device and moves across the touchscreen display device from the first location to the second location includes the first user input contacting a control element and moving a displayed position of the control element from the first location to the second location; and
the control element is distinct from the one or more graphical elements.

47. The computing system of claim 24, wherein:
the one or more first graphical elements do not move in a same direction and amount as the control element, responsive to the first user input moving the displayed position of the control element from the first location to the second location.

48. The computing system of claim 47, wherein:
the control element is presented along a displayed line that represents a path across which the first user input is able to move the control element.

49. The computing system of claim 47, wherein:
the first user input moves across at least a portion of the one or more first graphical elements, such that the control element is presented as superimposed over at least the portion of the one or more first graphical elements.

50. The computing system of claim 24, wherein:
receiving, by the computing system an indication that the first user input has released from the touchscreen display device at the third location;
receiving, by the computing system, user selection of an interface element to transition the touchscreen from presenting a first interface screen that includes the first statement to presenting a second interface screen; and
assigning, by the computing system, the third extent of user agreeableness with the first statement as the first user response to the first statement.

51. The computing system of claim 50, wherein:
presenting, by the computing system, a second statement on the second interface screen;
presenting, by the computing system, a second user response that includes same said first text indicating the first extent of user agreeableness with the first statement;
presenting, by the computing system, the one or more first graphical interface elements in the first configuration that graphically indicates the first extent of user agreeableness with the second statement;
receiving, by the computing system, an indication that second user input contacts the touchscreen display device and moves across the touchscreen display device from a fourth location to a fifth location while remaining in contact with the touchscreen display device;
changing, by the computing system, the presentation of the second user response from including the first text to including the second text indicating the second extent of user agreeableness with the first statement, responsive to the second user input having moved from the fourth location to the fifth location; and
changing, by the computing system, the presentation of the one or more first graphical interface elements from having the first configuration to having the second configuration that graphically indicates the second extent of user agreeableness with the second statement, responsive to the second user input having moved from the fourth location to the fifth location.
